SEQUENCE LISTING (22 sequences)

SEQ ID NO: 1 - RNA, 155 nt, synthetic construct
  misc_feature 1..155: note = 5' miRNA scaffold stem
  source 1..155: mol_type = other RNA
gtgctcgactagggataacagggtaattgtttgaatgaggcttcagtactttacagaatcgttgcctgcacatcttggaaacacttgctgggattacttcagctctttaacccaacagaaggctcgagagaaagcatatctgttgacagtgagcg

SEQ ID NO: 2 - RNA, 177 nt, synthetic construct
  misc_feature 1..177: note = 5' miRNA scaffold stem
  source 1..177: mol_type = other RNA
gtgctcgactagggataacagggtaattgtttgaatgaggcttcagtactttacagaatcgttgcctgcacatcttggaaacacttgctgggattacttcagctctttaacccaacagaaggctcgaggctagcgccgatataacggcggagaaagcatatctgttgacagtgagcg

SEQ ID NO: 3 - RNA, 155 nt, synthetic construct
  misc_feature 1..155: note = 5' miRNA scaffold stem
  source 1..155: mol_type = other RNA
gtgctcgactagggataacagggtaattgtttgaatgaggcttcagtactttacagaatcgttgcctgcacatcttggaaacacttgctgggattacttcacctgtttaacccaacagaaggcttaagagaaagcatatctgttgacagtgagcg

SEQ ID NO: 4 - RNA, 155 nt, synthetic construct
  misc_feature 1..155: note = 5' miRNA scaffold stem
  source 1..155: mol_type = other RNA
gtgctcgactagggataacagggtaattgtttgaatgaggcttcagtactttacagaatcgttgcctgcacatcttggaaacacttgctgggattacttcgacttttctaaccaacagaaggcgtacgagaaagcatatctgttgacagtgagcg

SEQ ID NO: 5 - RNA, 155 nt, synthetic construct
  misc_feature 1..155: note = 5' miRNA scaffold stem
  source 1..155: mol_type = other RNA
gtgctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcg

SEQ ID NO: 6 - RNA, 60 nt, synthetic construct
  misc_feature 1..60: note = 5' miRNA scaffold stem
  source 1..60: mol_type = other RNA
ctcgagccggatcaacgccctaggtttatgtttggatgaactgacatccgcgtatccgtc

SEQ ID NO: 7 - RNA, 32 nt, synthetic construct
  misc_feature 1..32: note = 5' miRNA scaffold stem
  source 1..32: mol_type = other RNA
ctcgagcagccagctttttgcgaatctcgaca

SEQ ID NO: 8 - RNA, 19 nt, synthetic construct
  misc_feature 1..19: note = miRNA scaffold loop
  source 1..19: mol_type = other RNA
tagtgaagccacagatgta

SEQ ID NO: 9 - RNA, 21 nt, synthetic construct
  misc_feature 1..21: note = miRNA scaffold loop
  source 1..21: mol_type = other RNA
gtagtgaaatatatattaaac

SEQ ID NO: 10 - RNA, 15 nt, synthetic construct
  misc_feature 1..15: note = miRNA scaffold loop
  source 1..15: mol_type = other RNA
tgtgttttttttgaa

SEQ ID NO: 11 - RNA, 132 nt, synthetic construct
  misc_feature 1..132: note = 3' miRNA scaffold stem
  source 1..132: mol_type = other RNA
tgcctactgcctcggacttcaaggggctaagatctggcaattatcttgtttactaaaactgaataccttgctatctctttgatacatttttacaaagctgaattaaaatggtataaattaaatcactttttt

SEQ ID NO: 12 - RNA, 132 nt, synthetic construct
  misc_feature 1..132: note = 3' miRNA scaffold stem
  source 1..132: mol_type = other RNA
tgcctactgcctcggacttcaagggactagtagatctcaattatcttgtttactaaaactgaataccttgctatctctttgatacatttttacaaagctgaattaaaatggtataaattaaatcactttttt

SEQ ID NO: 13 - RNA, 132 nt, synthetic construct
  misc_feature 1..132: note = 3' miRNA scaffold stem
  source 1..132: mol_type = other RNA
tgcctactgcctcggacttcaaggggctaaccggtggcaattatcttgtttactaaaactgaataccttgctatctctttgatacatttttacaaagctgaattaaaatggtataaattaaatcactttttt

SEQ ID NO: 14 - RNA, 131 nt, synthetic construct
  misc_feature 1..131: note = 3' miRNA scaffold stem
  source 1..131: mol_type = other RNA
tgcctactgcctcggacttcaaggggctattcgaagcaattatcttgtttactaaaactgaataccttgctatctctttgatacatttttacaaagctgaattaaaatggtataaattaaatcactttttt

SEQ ID NO: 15 - RNA, 132 nt, synthetic construct
  misc_feature 1..132: note = 3' miRNA scaffold stem
  source 1..132: mol_type = other RNA
tgcctactgcctcggacttcaaggggctagaattcggcaattatcttgtttactaaaactgaataccttgctatctctttgatacatttttacaaagctgaattaaaatggtataaattaaatcactttttt

SEQ ID NO: 16 - RNA, 54 nt, synthetic construct
  misc_feature 1..54: note = 3' miRNA scaffold stem
  source 1..54: mol_type = other RNA
tacggtaacgcggaatacgcaactattttatcaattttttgcgtcgacagactc

SEQ ID NO: 17 - RNA, 30 nt, synthetic construct
  misc_feature 1..30: note = 3' miRNA scaffold stem
  source 1..30: mol_type = other RNA
tcgcgattcgctttttcgtctttgagatct

SEQ ID NO: 18 - RNA, 56 nt, synthetic construct
  misc_feature 1..56: note = synthetic intron with splice donor
  source 1..56: mol_type = other RNA
ggaacggtgcattggaacgcggattccccgtgccaagagtgacgtaagtaccgcct

SEQ ID NO: 19 - RNA, 155 nt, synthetic construct
  misc_feature 1..155: note = synthetic intron with splice acceptor
  source 1..155: mol_type = other RNA
atagagtctataggcccacccccttggcttcgttagaacgcggctacaattaatacataaccttatgtatcatacacatacgatttaggtgacactatagaataacatccactttgcctttctctccacaggtgtccactcccaggtccaactgc

SEQ ID NO: 20 - RNA, 18 nt, synthetic construct
  misc_feature 1..18: note = synthetic intron with splice donor
  source 1..18: mol_type = other RNA
cagttcgaagaggtaagt

SEQ ID NO: 21 - RNA, 42 nt, synthetic construct
  misc_feature 1..42: note = synthetic intron with splice acceptor
  source 1..42: mol_type = other RNA
tactaactcttctttttttttttcacaggaccatcgatcgaa

SEQ ID NO: 22 - RNA, 18 nt, synthetic construct
  misc_feature 1..18: note = spacer sequence
  source 1..18: mol_type = other RNA
ccgccgatataacggcgg

(19) United States
(12) Patent Application Publication
(10) Pub. No.: US 2025/0313878 A1
(43) Pub. Date: Oct. 9, 2025

(54) IMPROVED PROTEIN PRODUCTION USING miRNA TECHNOLOGY

(71) Applicant: Novartis AG, Basel (CH)

(72) Inventors: David AUSLAENDER, Weil am Rhein (DE); Thomas JOSTOCK, Neuenburg am Rhein (DE)

(21) Appl. No.: 18/866,727

(22) PCT Filed: May 17, 2023
(86) PCT No.: PCT/IB2023/055054

§ 371 Date: Nov. 18, 2024

Related U.S. Application Data
(60) Provisional application No. 63/343,876, filed on May 19, 2022.

(51) Int. Cl. C12P 21/00; C12N 15/113; C12N 15/85

(52) U.S. Cl. CPC C12P 21/00; C12N 15/1137; C12N 15/85; C12N 2310/141; C12N 2310/531; C12N 2320/00; C12Y 304/00

(57) ABSTRACT

The present invention pertains to the use of miRNA technology for improving recombinant production of polypeptides of interest in host cells. Expression cassettes are provided which produce a miRNA targeting and down-regulating a host cell protein which interferes with production of the polypeptide of interest.

### FIELD OF THE INVENTION

The present invention pertains to the field of recombinant protein production. The present invention provides methods and means for reducing expression of host cell proteins which interfere with the production of the protein of interest using artificial miRNAs targeting the host cell proteins. In particular, expression cassettes for respective miRNAs comprising an intronic sequence with a template of a pri-miRNA, vectors and host cells comprising said expression cassettes, and their use for producing a protein of interest are provided.

### BACKGROUND OF THE INVENTION

The generation of recombinant cell lines for production of secreted proteins requires the transfection of a DNA vector into host cells and uses selection markers to enrich stable transfectants. The secreted proteins may affect cell parameters, such as growth, viability and/or productivity, which often requires cell line engineering methods to achieve high-expressing stable cell lines.

Similarly, the quality of the secreted protein can be affected by intrinsic cell-derived factors. Often, an endogenously expressed gene, such as a cell surface receptor, an enzyme or a protease, can be identified as the root cause of the undesired effect (see, e.g., WO 2014/097113 A2). Respective undesired effects include enzymatic cleavage of the polypeptide chain of the recombinant protein, such as digestion of the entire protein or amino acid clipping, i.e. removal of one or several amino acids from the N or C terminal end of the protein of interest. Other effects are unwanted post-translational modifications—or removal of desired modifications. Furthermore, endogenous gene products of the host cell may specifically interact with the protein of interest. Such an interaction may recruit the protein of interest from the supernatant of the cell culture, render it difficult to remove the endogenous protein during purification, or even lead to activation of signaling pathways in the host cells which result in reduced growth, viability and/or productivity of the cells. In other cases, the endogenous gene product might simply have chemical and physical properties which are highly similar to the protein of interest which makes it hard to develop a purification process which efficiently removes the host cell protein without diminishing the yield of the protein of interest. This is especially relevant for therapeutic proteins where a high purity and low residual levels of host cell proteins in the final product are a prerequisite for obtaining and maintaining marketing authorization.

Thus, endogenous gene products of the host cell may significantly interfere with the recombinant production of a protein of interest. In view of the above, there is a need to provide strategies to reduce unwanted influence of host cell proteins on the recombinant production of proteins of interest.

### SUMMARY OF THE INVENTION

The present inventors have found that the use of certain expression cassettes coding for an artificial miRNA are highly effective in specifically targeting a host cell protein which interferes with the production of a polypeptide of interest. Using this approach, expression of the interfering host cell protein can be significantly reduced and thereby, the yield and/or purity of the polypeptide of interest can be improved. Reduction of the interfering endogenous gene product using miRNA technology according to the present invention only requires minimal genetic engineering. The pri-miRNA template sequence can easily be introduced into already available plasmids for expression of the polypeptide of interest or standard vectors can be used to introduce the miRNA template into already established production host cells.

In addition, knockdown of the interfering host cell protein to a residual expression level in most cases is sufficient to significantly improve production of the polypeptide of interest. A complete knockout of the interfering gene product of the host cell, as done for example by genetic engineering of the host cell's genome, is much more complicated and might negatively affect the viability and efficacy of the host cells.

Therefore, in a first aspect, the present invention is directed to an expression cassette for expression of a miRNA in a host cell, comprising an intronic sequence comprising a template sequence for a pri-miRNA, wherein the pri-miRNA is suitable to be processed in the host cell to form a miRNA targeting a gene product of the host cell which interferes with the production of and/or modulates a polypeptide of interest recombinantly expressed in the host cell; and wherein the miRNA comprises a passenger strand and a guide strand having an artificial sequence.

In a second aspect, the present invention provides a vector nucleic acid for transfection of a host cell, comprising the expression cassette according to the first aspect.

In a third aspect, the present invention provides a host cell comprising the expression cassette according to the first aspect or the vector nucleic acid according to the second aspect, wherein the host cell is capable of recombinantly expressing the polypeptide of interest.

In a fourth aspect, the present invention provides a method for producing a polypeptide of interest in a host cell, comprising the steps of
(a) providing a host cell according to the third aspect;
(b) cultivating the host cell in a cell culture under conditions which allow for the expression of said polypeptide of interest;
(c) obtaining said polypeptide of interest from the cell culture; and
(d) optionally processing the polypeptide of interest;
   wherein the polypeptide of interest may optionally be encoded on the same vector nucleic acid, especially within the same expression cassette, as the pri-miRNA.

In a fifth aspect, the present invention provides a method for producing a host cell according to the third aspect, comprising the steps of
(a) introducing a vector nucleic acid according to the second aspect into a host cell, wherein the vector nucleic acid comprises a coding sequence for the polypeptide of interest, either within the expression cassette which expresses the miRNA, or within a further expression cassette; or
(b) introducing a vector nucleic acid according to the second aspect into a host cell, wherein the vector nucleic acid does not comprise a coding sequence for the polypeptide of interest, and introducing a further vector nucleic acid suitable for recombinant expression of the polypeptide of interest into the host cell, wherein the different vector nucleic acids may be introduced into the host cell simultaneously or consecutively, in any order.

In a sixth aspect, the present invention provides the use of the expression cassette according to the first aspect or the vector nucleic acid according to the second aspect or the host cell according to the third aspect for the production of a polypeptide of interest.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping. In specific embodiments, a nucleic acid refers to a double-stranded deoxyribonucleic acid.

The term “expression cassette” in particular refers to a nucleic acid construct which is capable of enabling and regulating the expression of a coding nucleic acid sequence and/or template nucleic acid sequence introduced therein. An expression cassette may comprise promoters, ribosome binding sites, enhancers and other control elements which regulate transcription of a gene or translation of an mRNA. The exact structure of an expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences. Some expression cassettes are only used for transcription of a template nucleic acid sequence into an RNA product such as a pri-miRNA. Such expression cassettes do not necessarily comprise regulatory elements for translation.

A template nucleic acid is understood according to this application as a DNA which is transcribed into a functional RNA product or a precursor thereof, especially a pri-miRNA. A functional RNA product in particular has a biological activity, alone or in combination with other RNA products and/or proteins, such as the activity of a miRNA (in combination with the proteins of the RISC) to interfere with expression of a target gene.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA. The vector according to the present invention may be present in circular or linearized form. A “vector nucleic acid” as used herein is a nucleic acid which forms a vector or is the nucleic acid part of a vector.

The terms “5′” and “3′” is a convention used to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left hand side, and 3′ (downstream) indicates genetic elements positioned towards the right hand side, when following this convention.

A “polypeptide” or “polypeptide chain” refers to a molecule comprising a polymer of amino acids linked together by peptide bonds. Polypeptides include polypeptides of any length, including proteins (for example, having more than 50 amino acids) and peptides (for example, having 2-49 amino acids). Especially, a polypeptide or polypeptide chain can be a part of a protein which consists of two or more polypeptide chains. Polypeptides include proteins and/or peptides of any activity or bioactivity. The polypeptide can be a pharmaceutically or therapeutically active compound, or a research tool to be utilized in assays and the like.

A target amino acid sequence is “derived” from or “corresponds” to a reference amino acid sequence if the target amino acid sequence shares an identity over its entire length with the reference amino acid sequence of at least 75%, more preferably at least 80%, at least 85%, at least 90%, at least 93%, at least 95%, at least 97%, at least 98% or at least 99%. In particular embodiments, a target amino acid sequence which is “derived” from or “corresponds” to a reference amino acid sequence is 100% identical over its entire length with the reference amino acid sequence. Similarly, a target nucleotide sequence is “derived” from or “corresponds” to a reference nucleotide sequence if the target nucleotide sequence shares an identity over its entire length with the reference nucleotide sequence of at least 75%, more preferably at least 80%, at least 85%, at least 90%, at least 93%, at least 95%, at least 97%, at least 98% or at least 99%. In particular embodiments, a target nucleotide sequence which is “derived” from or “corresponds” to a reference nucleotide sequence is 100% identical over its entire length with the reference nucleotide sequence. An “identity” of an amino acid sequence or nucleotide sequence is preferably determined according to the invention over the entire length of the reference sequence.

As used herein, a “microRNA”, abbreviated “miRNA”, is a single-stranded non-coding RNA molecule which plays a role in RNA silencing and post-transcriptional regulation of gene expression. miRNAs generally consist of 19 to 24 nucleotides, in particular about 22 nucleotides, especially 22 nucleotides. miRNA molecules are capable of silencing mRNAs comprising a complementary nucleotide sequence. Silencing of the target mRNA may occur by cleavage of the mRNA, destabilization of the mRNA or blockage of translation of the mRNA. Silencing of a target mRNA results in reduced or abolished production of the protein encoded by the target mRNA. It is generally understood in the art that the miRNA associates with dicer and argonaute proteins, forming an RNA-induced silencing complex (RISC) which binds to the target mRNA.

miRNAs are produced by transcription of a template DNA sequence into a miRNA precursor (pri-miRNA). The pri-miRNA contains a hairpin stem-loop structure with a double-stranded stem connected to a loop on one side and flanked by single-stranded 5′ and 3′ extensions on the other side. The double-stranded stem contains the guide strand, which forms the miRNA once processed, and the passenger strand which is essentially complementary to the guide strand. Especially, the passenger strand and the guide strand are complementary to each other except for the nucleotide pair at the end of the hairpin stem-loop structure, i.e. the nucleotide pair of passenger and guide strand which is furthest from the loop structure. Guide strand and passenger strand generally each have a length of about 19 to 24 nucleotides, especially of 22 nucleotides. The remaining parts of the pri-miRNA are referred to herein as miRNA scaffold. From 5′ to 3′, the pri-miRNA thus comprises (i) the 5′ miRNA scaffold stem, consisting of the 5′ single-stranded extension and the 5′ part of the stem structure up to the passenger strand; (ii) the passenger strand; (iii) the miRNA scaffold loop; (iv) the guide strand; and (v) the 3′ miRNA scaffold stem, consisting of the 3′ part of the stem structure following the guide strand and the 3′ single-stranded extension. Positions of the passenger strand and the guide strand may also be switched.

The pri-miRNA is processed by cleaving off the 5′ and 3′ miRNA scaffold stems, resulting in a hairpin structure termed pre-miRNA. From 5′ to 3′, the pre-miRNA consists of the passenger strand, the miRNA scaffold loop, and the guide strand; wherein the positions of the passenger strand and the guide strand may also be switched. Then the loop structure is cleaved off and the resulting RNA duplex is separated into the two single-stranded RNA molecules, the guide strand and the passenger strand. The guide strand which is complementary to the targeted mRNA molecule forms the RISC, while the passenger strand generally does not have any function.

The cells referred to herein in particular are host cells. According to the invention, the term “host cell” relates to any cell which can be transformed or transfected with an exogenous nucleic acid. Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell. A host cell in particular refers to a cell present in cell culture, especially a cell not present in a living multicellular organism.

The term “pharmaceutical composition” or “pharmaceutical formulation” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

The numbers given herein may in certain embodiments be understood as approximate numbers. In particular, the numbers preferably may be up to 10% higher and/or lower, in particular up to 9%, 8%, 7%, 6%, 5%, 4%, 3%, 2% or 1% higher and/or lower. In specific embodiments, the umbers given herein are not approximate numbers and may only vary within the inaccuracy of the technical measurement.

Numeric ranges described herein are inclusive of the numbers defining the range. The headings provided herein are not limitations of the various aspects or embodiments of this invention which can be read by reference to the specification as a whole. According to one embodiment, subject-matter described herein as comprising certain steps in the case of methods or as comprising certain ingredients in the case of compositions refers to subject-matter consisting of the respective steps or ingredients. It is preferred to select and combine preferred aspects and embodiments described herein and the specific subject-matter arising from a respective combination of preferred embodiments also belongs to the present disclosure.

### DETAILED DESCRIPTION OF THE INVENTION

The present invention is based on the development of new vector constructs which are used for knock-down of target host cell proteins. Endogenous proteins of host cells sometimes interfere with the production of a protein of interest in said host cell. In order to prevent this interference, the respective host cell protein(s) has to be removed from the host cell system. Often, gene knockouts were performed for this purpose, which is very time-consuming, labor-intensive and costly. The present invention differs from this approach as it acts on the RNA level leading to a knock-down of the mRNA coding for the interfering host cell protein. Generally, a complete knockout of the respective host cell gene is not required as a minimal expression should result in a similar desired phenotype. In addition, a knockout might lead to undesired cellular compensation effects. Therefore, a strong knock-down represents the preferred technology to deactivate an interfering host cell gene. In the past, shRNA molecules were encoded on a separate vector and expressed using strong polymerase-III promoters. With the new approach according to the present invention, normal polymerase-II promoters are sufficient and the pri-miRNA can be present on the same vector and even within the same expression cassette as the protein of interest which is produced in the host cell. This approach also showed high knock-down efficiency already on pool level during development of production cell lines which enables an extremely fast evaluation of the knock-down efficacy.

1. Expression Cassettes for Expression of a miRNA

In view of the above, in a first aspect the present invention provides an expression cassette for expression of a miRNA in a host cell, comprising an intronic sequence comprising a template sequence for a pri-miRNA, wherein the pri-miRNA is suitable to be processed in the host cell to form a miRNA targeting a gene product of the host cell which interferes with the production of and/or modulates a polypeptide of interest recombinantly expressed in the host cell; and wherein the miRNA comprises a passenger strand and a guide strand having an artificial sequence.

This expression cassette can be used to knock-down the targeted gene product of the host cell. Thereby, interference of said gene product with the recombinant production of the polypeptide of interest and/or modulation of the polypeptide of interest can be reduced or prevented. The miRNA can specifically target any host cell gene product which hampers production of the polypeptide of interest in its desired form. The gene product of the host cell targeted by the miRNA is generally referred to herein as “interfering gene product”.

It is understood that the miRNA produced from the expression cassette is at least partially complementary to and is capable of binding to and initiating silencing of the RNA “underlying” the interfering gene product. Hence, in embodiments where the interfering gene product is or comprises a protein or polypeptide, the underlying RNA in particular is the mRNA or pre-mRNA encoding the interfering gene product. In embodiments where the interfering gene product is or comprises a RNA, the underlying RNA in particular is said RNA or a precursor thereof. Silencing may occur via degradation of the targeted RNA or preventing the targeted mRNA from being translated.

Since the expression cassette comprises the template sequence for the pri-miRNA within an intronic sequence, it may contain further sequences for expression of other products, such as coding sequences for the production of the polypeptide of interest, coding sequences for the production of selectable marker, and template sequence for other RNA products, especially other pri-miRNAs. Alternatively, the expression cassette may be used exclusively for production of the miRNA targeting the interfering gene product.

### 1.1 Elements of the Expression Cassette

The expression cassette comprises the template sequence for the pri-miRNA within an intronic sequence. Upon expression, a pre-mRNA is formed which contains the intronic sequence. The intronic sequence is then spliced out of the pre-mRNA, thereby forming the pri-miRNA which thereafter is further processed to ultimately provide the miRNA. The formed pre-mRNA does not have to comprise any sequences coding for a polypeptide.

In certain embodiments, the expression cassette further comprises a polymerase II promoter. This promoter is functionally linked to the template sequence for the pri-miRNA and controls expression of the pri-miRNA. The promoter may be any RNA polymerase II promoter suitable for expression of a gene in a host cell, especially the host cell used for expression of the polypeptide of interest. In certain embodiments, the promoter is suitable for expression in a eukaryotic host cell, in particular a mammalian host cell, such as a CHO cell. For example, the promoter may be selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter, chicken β-actin promoter (CBA), β-actin promoter coupled with CMV early enhancer (CAGG), α-1-antitrypsin promoter, and inducible promoters such as tetracycline-inducible promoters (e.g. pTRE), and vanillic acid inducible promoters. In specific embodiments, the promoter is a CMV promoter or a SV40 promoter, especially a CMV promoter.

In certain embodiments, the expression cassette further comprises a terminator. The terminator is functionally linked to the template sequence for the pri-miRNA and controls expression of the pri-miRNA. The term “terminator” as used herein refers to a transcription terminator which terminates transcription of the DNA into RNA, especially by RNA polymerase II.

The template sequence for the pri-miRNA is in particular located between the promoter and the terminator of the expression cassette.

In certain embodiments, the expression cassette comprises a coding sequence encoding, for example, the polypeptide of interest or a selectable marker. In these embodiments, the expression cassette may further comprise a 5′ untranslated region (5′UTR) and a 3′ untranslated region (3′UTR). The intronic sequence comprising the template sequence for the pri-miRNA may be present within the 5′UTR, the 3′UTR or the coding sequence. In particular, the intronic sequence is present within the 5′UTR or the 3′UTR, especially within the 5′UTR. In alternative embodiments, the expression cassette does not comprise a coding sequence encoding a polypeptide.

The intronic sequence comprising the template sequence for the pri-miRNA in particular comprises a splice donor site upstream of the pri-miRNA and a corresponding splice acceptor site downstream of the pri-miRNA. With these splice donor and acceptor sites, the pri-miRNA is spliced out of the pre-mRNA after transcription.

In certain embodiments, the intronic sequence comprising two or more template sequences for a pri-miRNA. In these embodiments, the intronic sequence comprises a splice donor site upstream of the template sequence for the first pri-miRNA, i.e. the most 5′ template sequence, and a corresponding splice acceptor site downstream of the template sequence for the last pri-miRNA, i.e. the most 3′ template sequence. Adjacent template sequences within an intronic sequence may be separated from each other by a spacer sequence. Such a spacer sequence in particular forms a RNA stem loop structure, such as the sequence of SEQ ID NO: 22.

In certain embodiments, the expression cassette comprises only one intronic sequence with one or more template sequences for a pri-miRNA. In alternative embodiments, the expression cassette comprises two or more intronic sequence with one or more template sequences for a pri-miRNA.

The pri-miRNAs of the two or more template sequences present within the same or different intronic sequences in particular are different from each other. In specific embodiments, each miRNA produced from the pri-miRNAs targets a different interfering gene product. In alternative embodiments, each miRNA produced from the pri-miRNAs targets the same interfering gene product. In even further embodiments, some miRNAs produced from the pri-miRNAs target the same interfering gene product while other miRNAs produced from the pri-miRNAs target different interfering gene products. miRNAs targeting the same interfering gene product in particular bind to different parts of the RNA, especially the mRNA or pre-mRNA, of the interfering gene product.

The expression cassette may comprise a coding sequence which encodes a polypeptide. The coding sequence may in particular code for the polypeptide of interest or for a selectable marker. The coding sequence preferably is functionally linked to the polymerase II promoter and the terminator of the expression cassette. In embodiments wherein the expression cassette comprises the template sequence for the pri-miRNA and the coding sequence for the polypeptide of interest, the expression of these two elements are linked. Thereby, host cells which comprise the expression cassette and show a high expression level of the polypeptide of interest at the same time also have a high expression level of the miRNA. Clone development and selection is significantly simplified by this approach. In embodiments wherein the expression cassette comprises the template sequence for the pri-miRNA and the coding sequence for the selectable marker, the expression of the miRNA is linked to the selectable marker expression. Thus, by increasing the selection pressure during clone selection, also the miRNA level is increased.

The selectable marker may be selected from the group consisting of folate receptor (FAR), dihydrofolate reductase (DHFR), glutamine synthetase, puromycin, hygromycin, neomycin, zeocin, and blasticidin. In certain embodiments, the selectable marker is a folate receptor (FAR).

1.2 The miRNA

The expression cassette comprises a template sequence for the pri-miRNA. The pri-miRNA produced from the expression cassette may have any structure suitable for processing by the host cell in order to obtain a functional miRNA which targets the interfering gene product of the host cell. The functional miRNA in particular induces reduction of the level of the interfering gene product in the host cell.

In certain embodiments, the pri-miRNA comprises a passenger strand and a guide strand. The guide strand in particular comprises or consists of the miRNA formed after processing of the pri-miRNA by the host cell. The pri-miRNA furthermore, may comprise a miRNA scaffold loop and/or a miRNA scaffold stem, especially a 5′ miRNA scaffold stem and a 3′ miRNA scaffold stem. In specific embodiments, the pri-miRNA comprises, from 5′ to 3′, a 5′ miRNA scaffold stem, a passenger strand, a miRNA scaffold loop, a guide strand, and a 3′ miRNA scaffold stem. In alternative embodiments, the pri-miRNA comprises, from 5′ to 3′, a 5′ miRNA scaffold stem, a guide strand, a miRNA scaffold loop, a passenger strand, and a 3′ miRNA scaffold stem. Embodiments wherein the passenger strand is positioned upstream of the guide strand are preferred.

The passenger strand and the guide strand of the pri-miRNA have artificial sequences. An artificial sequence in this respect refers to a sequence which is not found as passenger or guide strand in naturally occurring miRNAs. In particular, the sequences of the passenger strand and the guide strand are not found in naturally occurring miRNAs. In certain embodiments, the guide strand comprises and in particular consists of a sequence which is designed to reduce the expression of the interfering gene product. Especially, the sequence of the guide strand is complementary to a part of the mRNA coding for the gene product of the host cell targeted by the miRNA.

In certain embodiments, one or more of the scaffold sequences of the pri-miRNA or pre-miRNA are derived from a naturally occurring pri-miRNA, especially a pri-miRNA naturally occurring in mammals, in particular in humans. In specific embodiments, all of the scaffold sequences of the pri-miRNA are derived from a naturally occurring pri-miRNA, especially a pri-miRNA naturally occurring in mammals, in particular in humans. In particular, all of the scaffold sequences of the pri-miRNA are derived from the same naturally occurring pri-miRNA. The scaffold sequences of the pri-miRNA in particular comprise the 5′ miRNA scaffold stem, the miRNA scaffold loop and the 3′ miRNA scaffold stem. Suitable naturally occurring pri-miRNAs from which the scaffold sequences may be derived include miR-30A, miR-E, SIBR, eSIBR, miR-1, miR-155, miR-16, miR-16-1, miR-16-2, miR-3G, miRGE, miR100, miR125b, miR-130a, miR-190a, miR-193a, miR-211, miR-26a, miR-340, miR-7-2, miR-96, and miR-44. Thus, in one embodiments the 5′ miRNA scaffold stem, the miRNA scaffold loop, and the 3′ miRNA scaffold stem are derived from one or more pre-miRNAs selected from the group consisting of miR-30A, miR-E, SIBR, eSIBR, miR-1, miR-155, miR-16, miR-16-1, miR-16-2, miR-3G, miRGE, miR 100, miR 125b, miR-130a, miR-190a, miR-193a, miR-211, miR-26a, miR-340, miR-7-2, miR-96, and miR-44. In specific embodiments, the naturally occurring pri-miRNA from which the scaffold sequences are derived is miR-30A.

In specific embodiments, all of the scaffold sequences of the pri-miRNA share a nucleotide sequence identity with the corresponding scaffold sequences of a naturally occurring pri-miRNA of at least 80%, especially at least 90%, in particular at least 95% over their entire length. In certain embodiments, the 5′ miRNA scaffold stem of the pri-miRNA shares a nucleotide sequence identity with the corresponding scaffold sequence of a naturally occurring pri-miRNA of at least 80%, especially at least 85%, in particular at least 90% over its entire length. In certain embodiments, the 3′ miRNA scaffold stem of the pri-miRNA shares a nucleotide sequence identity with the corresponding scaffold sequence of a naturally occurring pri-miRNA of at least 80%, especially at least 90%, in particular at least 95% over its entire length. In certain embodiments, the miRNA scaffold loop of the pri-miRNA shares a nucleotide sequence identity with the corresponding scaffold sequence of a naturally occurring pri-miRNA of at least 60%, especially at least 70%, in particular at least 75% over its entire length. In these embodiments, the naturally occurring pri-miRNA may in particular be miR-30A.

In certain embodiments, the 5′ miRNA scaffold stem of the pri-miRNA comprises the nucleotide sequence of any one of SEQ ID NOs: 1-7 or a sequence derived therefrom. In particular, the 5′ miRNA scaffold stem of the pri-miRNA comprises the nucleotide sequence of any one of SEQ ID NOs: 1-7 or a sequence sharing a nucleotide sequence identity therewith of at least 90%, preferably at least 95%, more preferably at least 98%, and most preferably 100%. Especially, the 5′ miRNA scaffold stem of the pri-miRNA consists of the nucleotide sequence of any one of SEQ ID NOs: 1-7, in particular any one of SEQ ID NOs: 1-4, especially SEQ ID NO: 1.

In certain embodiments, the miRNA scaffold loop of the pri-miRNA comprises the nucleotide sequence of any one of SEQ ID NOs: 8-10 or a sequence derived therefrom. In particular, the miRNA scaffold loop of the pri-miRNA comprises the nucleotide sequence of any one of SEQ ID NOs: 8-10 or a sequence sharing a nucleotide sequence identity therewith of at least 75%, preferably at least 85%, more preferably at least 90%, and most preferably 100%. Especially, the miRNA scaffold loop of the pri-miRNA consists of the nucleotide sequence of any one of SEQ ID NOs: 8-10, in particular SEQ ID NO: 8.

In certain embodiments, the 3′ miRNA scaffold stem of the pri-miRNA comprises the nucleotide sequence of any one of SEQ ID NOs: 11-17 or a sequence derived therefrom. In particular, the 3′ miRNA scaffold stem of the pri-miRNA comprises the nucleotide sequence of any one of SEQ ID NOs: 11-17 or a sequence sharing a nucleotide sequence identity therewith of at least 90%, preferably at least 95%, more preferably at least 98%, and most preferably 100%. Especially, the 3′ miRNA scaffold stem of the pri-miRNA consists of the nucleotide sequence of any one of SEQ ID NOs: 11-17, in particular any one of SEQ ID NOs: 11-14, especially SEQ ID NO: 11.

In certain embodiments, the template sequence for the pri-miRNA comprises at least one recognition site, especially two recognition sites, for a DNA restriction enzyme. In particular, the two recognition sites are for different DNA restriction enzymes and generate different overhangs after cleavage. The two recognition sites preferably flank the pre-miRNA part of the pri-miRNA-which comprises the guide strand, the passenger strand and the miRNA scaffold loop-on both sides. In particular, one of the recognition sites is located within the sequence which is transcribed into the 5′ miRNA scaffold stem and the other recognition site is located within the sequence which is transcribed into the 3′ miRNA scaffold stem. In specific embodiments, the recognition sites are located within the sequences which are transcribed into the single-stranded parts of the 5′ and 3′ miRNA scaffold stems. The recognition sites in particular are unique recognition sites within the expression cassette, especially within the entire vector harboring the expression cassette.

### 1.3 the Target Gene

The miRNA expressed by the expression cassette is for targeting a gene product of the host cell (“interfering gene product”) which interferes with the production of and/or modulates a polypeptide of interest recombinantly expressed in the host cell. The interfering gene product may be any gene product which interferes with the production of and/or modulates the polypeptide of interest. Exemplary interfering gene products are for example selected from the group consisting of
(i) proteases which are capable of cleaving the polypeptide of interest
(ii) proteins involved in posttranslational modification of the polypeptide of interest
(iii) receptors or binding partners of the polypeptide of interest
(iv) proteins which are difficult to separate from the polypeptide of interest
(v) proteins involved in folding and/or secretion of the polypeptide of interest
(vi) proteins involved in transport of components necessary for production or modification of the polypeptide of interest
(vii) proteins involved in degradation of the polypeptide of interest
(viii) proteins which share a sequence identity of at least 70%, in particular at least 80%, with the polypeptide of interest over its entire length, or endogenous homologues of the polypeptide of interest.

Expression of the miRNA reduces the production of the interfering gene product in the host cell and thereby decreases or eliminates interference with the production and/or modulation of the polypeptide of interest. The interfering gene product in particular is an endogenous gene product of the host cell.

In certain embodiments, the interfering gene product is a protease which is capable of cleaving the polypeptide of interest. In these embodiments, expression of the miRNA in particular reduces cleavage of the polypeptide of interest by the targeted protease.

In certain embodiments, the interfering gene product is a protein involved in posttranslational modification of the polypeptide of interest. For example, the interfering gene product may be a transferase which is capable of catalyzing post-translational modification of the polypeptide of interest. Exemplary post-translational modifications include acetylation, acylation, sulfation, phosphorylation, alkylation, hydroxylation, amidation, carboxylation, palmitoylation, myristoylation, and isoprenylation. In these embodiments, expression of the miRNA in particular reduces the amount of the post-translational modification catalyzed by the targeted transferase.

In further embodiments, the interfering gene product may be an enzyme which is capable of catalyzing the removal of a post-translational modification or of a chemical group of the polypeptide of interest. The interfering gene product may for example be a hydrolase such as a lipase, a phosphatase, or a glycosydase. In these embodiments, expression of the miRNA in particular increases the amount of the respective post-translational modification or chemical group which would be removed by the interfering gene product.

In further embodiments, the interfering gene product may be a protein involved in glycosylation of the polypeptide of interest. Exemplary proteins include glycosyltransferases, glycosidases and nucleotide sugar transporters, such as fucosyltransferases, and sialyltransferases. In these embodiments, expression of the miRNA in particular reduces the degree of glycosylation motifs generated by or with support of the interfering gene product.

In certain embodiments, the interfering gene product is a receptor or binding partner of the polypeptide of interest. Receptors and binding partners include any gene products which bind to the polypeptide of interest and thereby interfere with its production. This in particular also includes gene products of other species, for example in embodiments where the host cell is not of the same species as the polypeptide of interest (e.g. expression of a human polypeptide in a CHO host cell), especially homologs of the natural receptor or binding partner of the polypeptide of interest. Receptors and binding partners may interfere with production of the polypeptide of interest, for example, by sequestering the polypeptide of interest from the cell culture medium, thereby reducing its yield in the culture supernatant, or by decreasing host cell proliferation or survival because binding of the polypeptide of interest to the receptor or binding partner activates a signal pathway in the host cells.

In certain embodiments, the interfering gene product is a protein which is difficult to separate from the polypeptide of interest. Respective interfering gene products for example include gene products which bind to the polypeptide of interest and gene products which have chemical and/or physical properties which are similar to those of the polypeptide of interest. Exemplary chemical and/or physical properties in this respect are molecular size, overall charge, charge distribution, pl value, hydrophobicity, and binding to capture ligands such as protein A or protein G. In these embodiments, the interfering gene product in particular cannot be separated well from the polypeptide of interest using standard purification methods. For example, the interfering gene product and the polypeptide of interest may have similar binding and/or elution characteristics on one or more chromatography matrices. In such embodiments, removal of the interfering gene product from the product solution results in significant loss of the polypeptide of interest and thus, in low yield of the purified polypeptide of interest.

### 2. Vectors and Host Cells

In a second aspect the present invention provides a vector nucleic acid for transfection of a host cell, comprising the expression cassette according to the first aspect. The vector may be any vector suitable for transfection of the host cell. In certain embodiments, the vector is a plasmid. In other embodiments, the vector is a viral vector.

The vector nucleic acid may comprise further elements in addition to the expression cassette. For example, the vector nucleic acid may comprise an origin of replication (ORI), a coding sequence encoding the polypeptide of interest, a selectable marker gene, and/or an antibiotics resistance gene.

In certain embodiments, the vector nucleic acid does not comprise a coding sequence encoding the polypeptide of interest. In specific embodiments where the vector nucleic acid does not comprise a coding sequence encoding the polypeptide of interest, the expression cassette according to the first aspect of the invention does not comprise a coding sequence for a polypeptide. In these embodiments, the vector nucleic acid in particular comprises a further expression cassette comprising a selectable marker gene. In alternative embodiments where the vector nucleic acid does not comprise a coding sequence encoding the polypeptide of interest, the expression cassette according to the first aspect of the invention comprises a coding sequence which encodes a selectable marker.

In certain embodiments, the vector nucleic acid comprises a coding sequence encoding the polypeptide of interest. The coding sequence encoding the polypeptide of interest may be present within the expressing cassette according to the first aspect of the invention or may be present in a further expression cassette. In specific embodiments, the vector nucleic acid comprises at least two expression cassettes, a first expression cassette for expression of the polypeptide of interest and a second expression cassette for the expression of a selectable marker, with either the first or the second expression cassette being an expressing cassette according to the first aspect of the invention. In alternative embodiments, the vector nucleic acid comprises at least three expression cassettes, a first expression cassette being an expressing cassette according to the first aspect of the invention, a second expression cassette for expression of the polypeptide of interest, and a third expression cassette for the expression of a selectable marker. In case the polypeptide of interest is comprised of two or more different polypeptide chains, the different polypeptide chains may be encoded within the same expression cassette or within separate expression cassettes. The additional expression cassettes comprising coding sequences of different polypeptide chains of the polypeptide of interest may also be present on the vector nucleic acid.

In certain embodiments, two or more of the expression cassettes of the vector nucleic acid are expression cassettes according to the first aspect of the invention. These expression cassettes may each comprise template sequences for the same or different pri-miRNAs, in particular for different pri-miRNAs, which may target the same of different interfering gene products of the host cell.

In a third aspect, the present invention provides a host cell comprising the expression cassette according to the first aspect or the vector nucleic acid according to the second aspect, wherein the host cell is capable of recombinantly expressing the polypeptide of interest.

The host cell may be any type of host cell which produces a gene product which interferes with the production of and/or modulates a polypeptide of interest. In certain embodiments, the host cell is known for producing the interfering gene product. The host cell is in particular suitable to produce the polypeptide of interest.

A host cell “capable of recombinantly expressing the polypeptide of interest” in particular is a host cell which comprises a nucleic acid encoding for the polypeptide of interest. The nucleic acid encoding for the polypeptide of interest in particular is heterologous to the host cell and especially was introduced into the host cell using genetic engineering techniques.

In certain embodiments, the host cell is a mammalian cell. The host cell may in particular be a rodent cell, primate cell or a human cell. In certain embodiments, the mammalian cell is selected from, but not limited to, the group consisting of cells derived from mice, such as COP, L, C127, Sp2/0, NS0, NS1, At20 and NIH3T3; rats, such as PC12, PC12h, GH3, MIT, YB2/0 and Y0; hamsters, such as BHK, CHO and DHFR gene defective CHO; monkeys, such as COS1, COS3, COS7, CV1 and Vero; and humans, such as Hela, HEK293, CAP, retina-derived PER-C6, cells derived from diploid fibroblasts, myeloma cells and HepG2. In specific embodiments, the host cell is a Chinese hamster ovary (CHO) cell. The host cell may be suitable for suspension cultures and/or adherent cultures, and in particular can be used in suspension cultures.

The host cell may contain further exogenous nucleic acids in addition to the expression cassette according to the first aspect or the vector nucleic acid according to the second aspect of the invention. In particular, the host cell may contain an expression cassette for expression of the polypeptide of interest which is not the expression cassette according to the first aspect and which is not present on the vector nucleic acid according to the second aspect. Said expression cassette for expression of the polypeptide of interest may be present on a further vector nucleic acid or integrated into the genome of the host cell.

In certain embodiments, the coding sequence of the polypeptide of interest is present in the host cell
(i) within the expression cassette which expresses the miRNA,
(ii) within a further expression cassette on the same vector nucleic acid as the expression cassette which expresses the miRNA, or
(iii) on a further vector nucleic acid or in the genome of the host cell.

### 3. Production Methods

In a fourth aspect, the present invention provides a method for producing a polypeptide of interest in a host cell, comprising the steps of
(a) providing a host cell according to the third aspect;
(b) cultivating the host cell in a cell culture under conditions which allow for the expression of said polypeptide of interest;
(c) obtaining said polypeptide of interest from the cell culture; and
(d) optionally processing the polypeptide of interest;
   wherein the polypeptide of interest may optionally be encoded on the same vector nucleic acid, especially within the same expression cassette, as the pri-miRNA.

The steps of the method are generally performed in the indicated order.

In certain embodiments, the method further comprises between steps (a) and (b) the steps of
(a1) inoculating a cell culture medium with the host cell to provide a cell culture, and
(a2) cultivating the host cell in the cell culture under conditions which allow for increasing the number of cells in the cell culture.

Suitable conditions for cultivating the host cells, increasing their cell number and expressing the polypeptide of interest depend on the specific host cell, vector and expression cassette used in the method. The skilled person can readily determine suitable conditions and they are also already known in the art for a plurality of host cells. In certain embodiments, the vector nucleic acid in the host cell comprises one or more selectable marker genes. In these embodiments, the culturing conditions in step (a2) and/or (b) may include the presence of corresponding selection agent(s) in the cell culture medium.

Obtaining the polypeptide of interest from the cell culture in step (c) in particular includes isolating the polypeptide of interest from the cell culture. Isolation of the polypeptide of interest in particular refers to the separation of the polypeptide of interest from the remaining components of the cell culture. The term “cell culture” as used herein in particular includes the cell culture medium and the cells. In certain embodiments, the polypeptide of interest is secreted by the host cell. In these embodiments, the polypeptide of interest is isolated from the cell culture medium. Separation of the polypeptide of interest from the cell culture medium may be performed, for example, by chromatographic methods. Suitable methods and means for isolating the polypeptide of interest are known in the art and can be readily applied by the skilled person.

The obtained polypeptide of interest may optionally be subject to further processing steps (d) such as e.g. further purification, modification and/or formulation steps in order to produce the polypeptide of interest in the desired quality and composition. Such further processing steps and methods are generally known in the art. Suitable purification steps for example include affinity chromatography, size exclusion chromatography, anion- and/or cation exchange chromatography, hydrophilic interaction chromatography and reverse phase chromatography. Further steps may include virus inactivation, ultrafiltratrion and diafiltration. Formulation steps may include buffer exchange, addition of formulation components, pH adjustment, and concentration adjustment. Any combination of these and further steps may be used.

In certain embodiments, the method for producing the polypeptide of interest further comprises as step (d) or part of step (d) the step of providing a pharmaceutical formulation comprising the polypeptide of interest. Providing a pharmaceutical formulation comprising the polypeptide of interest or formulating the polypeptide of interest as a pharmaceutical composition in particular comprises exchanging the buffer solution or buffer solution components of the composition comprising the polypeptide of interest. Furthermore, this step may include lyophilization of the polypeptide of interest. In particular, the polypeptide of interest is transferred into a composition only comprising pharmaceutically acceptable ingredients.

In certain embodiments, the production of the interfering gene product in the host cell is reduced compared to the same host cell not expressing the miRNA targeting the interfering gene product. Especially, the amount of interfering gene product in the host cell is reduced to 50% or less, in particular 20% or less or even 10% or less compared the same host cell not expressing the miRNA targeting the interfering gene product.

The present invention further provides a method of producing a polypeptide of interest with increased yield and/or increased purity, comprising the steps of
(a) providing a host cell according to the third aspect;
(b) cultivating the host cell in a cell culture under conditions which allow for the expression of said polypeptide of interest;
(c) obtaining said polypeptide of interest from the cell culture; and
(d) optionally processing the polypeptide of interest;
   wherein the polypeptide of interest may optionally be encoded on the same vector nucleic acid, especially within the same expression cassette, as the pri-miRNA.

The increase in yield and/or purity is determined in comparison with the same method for producing the polypeptide of interest, wherein the host cell does not comprise an expression cassette according to the first aspect of the present invention and does not produce a miRNA targeting the interfering gene product of the host cell.

The present invention further provides a method of increasing the yield and/or increasing the purity of a polypeptide of interest produced by a host cell, comprising the steps of
(a1) providing a host cell capable of producing the polypeptide of interest;
(a2) introducing a vector nucleic acid according to the second aspect of the present invention into the host cell;
(b) cultivating the host cell in a cell culture under conditions which allow for the expression of said polypeptide of interest;
(c) obtaining said polypeptide of interest from the cell culture; and
(d) optionally processing the polypeptide of interest.

In certain embodiments, the vector nucleic acid introduced into the host cell in step (a2) does not comprise a coding sequence for the polypeptide of interest.

In a fifth aspect, the present invention provides a method for producing a host cell according to the third aspect, comprising the steps of
(a) introducing a vector nucleic acid according to the second aspect into a host cell, wherein the vector nucleic acid comprises a coding sequence for the polypeptide of interest, either within the expression cassette which expresses the miRNA, or within a further expression cassette; or
(b) introducing a vector nucleic acid according to the second aspect into a host cell, wherein the vector nucleic acid does not comprise a coding sequence for the polypeptide of interest, and introducing a further vector nucleic acid suitable for recombinant expression of the polypeptide of interest into the host cell, wherein the different vector nucleic acids may be introduced into the host cell simultaneously or consecutively, in any order.

The vector nucleic acid is artificially introduced into the host cell. In particular, the vector nucleic acid is introduced by transfection. Transfection in this respect may be transient or stable, and especially stable transfection is used. Hence, in certain embodiments the produced host cell comprises the expression cassette according to the first aspect stably integrated into its genome.

In a sixth aspect, the present invention provides the use of the expression cassette according to the first aspect or the vector nucleic acid according to the second aspect or the host cell according to the third aspect for the production of a polypeptide of interest. The features and embodiments of the method for producing a polypeptide of interest described herein likewise apply to this use.

The present invention further provides the use of the expression cassette according to the first aspect or the vector nucleic acid according to the second aspect for improving production of a polypeptide of interest by a host cell, including introducing the expression cassette or vector nucleic acid into a host cell capable of producing the polypeptide of interest. In certain embodiments, the vector nucleic acid introduced into the host cell does not comprise a coding sequence for the polypeptide of interest. Improving production of a polypeptide of interest may include increasing the yield and/or increasing the purity of the polypeptide of interest.

The present invention further provides the use of the vector nucleic acid for the transfection of a host cell. In particular, the host cell is a mammalian cell such as a Chinese hamster ovary (CHO) cell.

### 4. Specific Embodiments

In the following, specific embodiments of the present invention are described. These embodiments can be combined with the further embodiments, features and examples described herein.

Embodiment 1 An expression cassette for expression of an miRNA in a host cell, comprising an intronic sequence comprising a template sequence for a pri-miRNA, wherein the pri-miRNA is suitable to be processed in the host cell to form a miRNA targeting a gene product of the host cell which interferes with the production of and/or modulates a polypeptide of interest recombinantly expressed in the host cell; and wherein the miRNA comprises a passenger strand and a guide strand having an artificial sequence.

Embodiment 2 The expression cassette according to embodiment 1, further comprising a polymerase II promoter and a terminator functionally linked to the template sequence for a pri-miRNA, wherein the template sequence for the pri-miRNA is located between promoter and terminator of the expression cassette.

Embodiment 3 The expression cassette according to embodiment 1 or 2, wherein the template sequence for the pri-miRNA is present within the 5′ untranslated region, the 3′ untranslated region or a coding sequence of the expression cassette, especially within the 5′ untranslated region or the 3′ untranslated region.

Embodiment 4 The expression cassette according to any one of embodiments 1 to 3, wherein the pre-mRNA produced upon transcription of the expression cassette comprises the pri-miRNA, and wherein the miRNA is formed by processing of the pre-mRNA.

Embodiment 5 The expression cassette according to any one of embodiments 1 to 4, comprising a splice donor site upstream of the pri-miRNA and a corresponding splice acceptor site downstream of the pri-miRNA.

Embodiment 6 The expression cassette according to any one of embodiments 1 to 5, comprising two or more template sequences for a pri-miRNA, each miRNA targeting the same or a different gene product.

Embodiment 7 The expression cassette according to embodiment 6, wherein the two or more template sequences for a pri-miRNA are located within the same intronic sequence.

Embodiments 8 The expression cassette according to embodiment 7, comprising a splice donor site upstream of the template sequence for the first pri-miRNA and a corresponding splice acceptor site downstream of the template sequence for the last pri-miRNA.

Embodiment 9 The expression cassette according to any one of embodiments 6 to 8, wherein adjacent template sequences of the two or more template sequences for a pri-miRNA are separated from each other by a spacer sequence which forms an RNA stem loop structure, such as the sequence of SEQ ID NO: 22.

Embodiment 10 The expression cassette according to any one of embodiments 1 to 9, wherein the pri-miRNA comprises, from 5′ to 3′, a 5′ miRNA scaffold stem, a passenger strand, a miRNA scaffold loop, a guide strand, and a 3′ miRNA scaffold stem, or wherein the pri-miRNA comprises, from 5′ to 3′, a 5′ miRNA scaffold stem, a guide strand, a miRNA scaffold loop, a passenger strand, and a 3′ miRNA scaffold stem.

Embodiment 11 The expression cassette according to embodiment 10, wherein the 5′ miRNA scaffold stem, the miRNA scaffold loop, and the 3′ miRNA scaffold stem are derived from one or more pri-miRNAs selected from the group consisting of miR-30A, miR-E, SIBR, eSIBR, miR-1, miR-155, miR-16, miR-16-1, miR-16-2, miR-3G, miRGE, miR-100, miR-125b, miR-130a, miR-190a, miR-193a, miR-211, miR-26a, miR-340, miR-7-2, miR-96, and miR-44.

Embodiment 12 The expression cassette according to embodiment 10 or 11, wherein the 5′ miRNA scaffold stem comprises the nucleotide sequence of any one of SEQ ID NOs: 1-4, and/or the miRNA scaffold loop comprises the nucleotide sequence of SEQ ID NO: 8, and/or the 3′ miRNA scaffold stem comprises the nucleotide sequence of any one of SEQ ID NOs: 11-14.

Embodiment 13 The expression cassette according to any one of embodiments 10 to 12, wherein the 5′ miRNA scaffold stem and the 3′ miRNA scaffold stem each comprises a recognition site for a restriction enzyme.

Embodiment 14 The expression cassette according to embodiment 13, wherein the recognition sites in the 5′ miRNA scaffold stem and the 3′ miRNA scaffold stem each are unique recognition sites.

Embodiment 15 The expression cassette according to any one of embodiments 1 to 14, wherein the gene product of the host cell targeted by the miRNA is selected from the group consisting of a protease which is capable of cleaving the polypeptide of interest, a protein involved in posttranslational modification of the polypeptide of interest, a receptor or binding partner of the polypeptide of interest, a protein which is difficult to separate from the polypeptide of interest, a protein involved in folding and/or secretion of the polypeptide of interest, a protein involved in transport of components necessary for production or modification of the polypeptide of interest, a protein involved in degradation of the polypeptide of interest, a protein which shares a sequence identity of at least 70%, in particular at least 80%, with the polypeptide of interest over its entire length, and an endogenous homologue of the polypeptide of interest.

Embodiment 16 The expression cassette according to any one of embodiments 1 to 15, wherein the gene product of the host cell targeted by the miRNA is a protease which is capable of cleaving the polypeptide of interest.

Embodiment 17 The expression cassette according to any one of embodiments 1 to 15, wherein the gene product of the host cell targeted by the miRNA is a transferase which is capable of catalyzing post-translational modification of the polypeptide of interest, for example acetylation, acylation, sulfation, phosphorylation, alkylation, hydroxylation, amidation, carboxylation, palmitoylation, myristoylation, and isoprenylation.

Embodiment 18 The expression cassette according to any one of embodiments 1 to 15, wherein the gene product of the host cell targeted by the miRNA is an enzyme which is capable of catalyzing the removal of a post-translational modification or of a chemical group of the polypeptide of interest, for example a hydrolase such as a lipase, a phosphatase, or a glycosydase.

Embodiment 19 The expression cassette according to any one of embodiments 1 to 15, wherein the gene product of the host cell targeted by the miRNA is a protein involved in glycosylation of the polypeptide of interest, in particular a glycosyltransferase, a glycosidase, or a nucleotide sugar transporter, for example a fucosyltransferase, or a sialyltransferase.

Embodiment 20 The expression cassette according to any one of embodiments 1 to 19, wherein the miRNA targets an endogenous gene product of the host cell. Embodiment 21 The expression cassette according to any one of embodiments 1 to 20, wherein the artificial sequence of the guide strand is complementary to a part of the mRNA coding for the gene product of the host cell targeted by the miRNA.

Embodiment 22 The expression cassette according to any one of embodiments 1 to 20, wherein the artificial sequence of the passenger strand and/or of the guide strand is not found in naturally occurring miRNAs.

Embodiment 23 The expression cassette according to any one of embodiments 1 to 22, wherein the promoter is selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter, chicken β-actin promoter (CBA), β-actin promoter coupled with CMV early enhancer (CAGG), α-1-antitrypsin promoter, and inducible promoters such as tetracycline-inducible promoters (e.g. pTRE), and vanillic acid inducible promoters.

Embodiment 24 The expression cassette according to any one of embodiments 1 to 23, wherein the promoter is a CMV promoter or a SV40 promoter.

Embodiment 25 The expression cassette according to any one of embodiments 2 to 24, further comprising a coding sequence for the polypeptide of interest, functionally linked to the polymerase II promoter and the terminator.

Embodiment 26 The expression cassette according to any one of embodiments 2 to 24, further comprising a coding sequence for a selectable marker, functionally linked to the polymerase II promoter and the terminator.

Embodiment 27 The expression cassette according to embodiment 26, wherein the selectable marker is selected from the group consisting of folate receptor (FAR), dihydrofolate reductase (DHFR), glutamine synthetase, puromycin, hygromycin, neomycin, zeocin, and blasticidin.

Embodiment 28 The expression cassette according to any one of embodiments 1 to 27, wherein the host cell is a mammalian cell, in particular a human, primate or rodent cell, especially a human or hamster cell.

Embodiment 29 The expression cassette according to any one of embodiments 1 to 28, wherein the host cell is a CHO cell.

Embodiment 30 A vector nucleic acid for transfection of a host cell, comprising the expression cassette according to any one of embodiments 1 to 29.

Embodiment 31 The vector nucleic acid according to embodiment 30, further comprising an additional expression cassette suitable for expressing the polypeptide of interest.

Embodiment 32 A host cell comprising the expression cassette according to any one of embodiments 1 to 29 or the vector nucleic acid according to embodiment 30 or 31, wherein the host cell is capable of recombinantly expressing the polypeptide of interest.

Embodiment 33 The host cell according to embodiment 31 or 32, being a mammalian cell, in particular a human, primate or rodent cell, especially a human or hamster cell.

Embodiment 34 The host cell according to embodiment 33, being a CHO cell.

Embodiment 35 The host cell according to any one of embodiments 32 to 34, wherein the coding sequence of the polypeptide of interest is present in the host cell
(i) within the expression cassette which expresses the miRNA,
(ii) within a further expression cassette on the same vector nucleic acid as the expression cassette which expresses the miRNA, or
(iii) on a further vector nucleic acid or in the genome of the host cell.

Embodiment 36 A method for producing a polypeptide of interest in a host cell, comprising the steps of
(a) providing a host cell according to any one of embodiments 32 to 35;
(b) cultivating the host cell in a cell culture under conditions which allow for the expression of said polypeptide of interest;
(c) obtaining said polypeptide of interest from the cell culture; and
(d) optionally processing the polypeptide of interest;
   wherein the polypeptide of interest may optionally be encoded on the same vector nucleic acid, especially within the same expression cassette, as the pri-miRNA.

Embodiment 37 The method according to embodiment 36, wherein step (d) comprises providing a pharmaceutical formulation comprising the polypeptide of interest.

Embodiment 38 A method of increasing the yield and/or increasing the purity of a polypeptide of interest produced by a host cell, comprising the steps of
(a1) providing a host cell capable of producing the polypeptide of interest;
(a2) introducing a vector nucleic acid according to embodiment 30 into the host cell;
(b) cultivating the host cell in a cell culture under conditions which allow for the expression of said polypeptide of interest;
(c) obtaining said polypeptide of interest from the cell culture; and
(d) optionally processing the polypeptide of interest.

Embodiment 39 A method for producing a host cell according to any one of embodiments 32 to 35, comprising the steps of
(a) introducing a vector nucleic acid according to the second aspect into a host cell, wherein the vector nucleic acid comprises a coding sequence for the polypeptide of interest, either within the expression cassette which expresses the miRNA, or within a further expression cassette; or
(b) introducing a vector nucleic acid according to the second aspect into a host cell, wherein the vector nucleic acid does not comprise a coding sequence for the polypeptide of interest, and introducing a further vector nucleic acid suitable for recombinant expression of the polypeptide of interest into the host cell, wherein the different vector nucleic acids may be introduced into the host cell simultaneously or consecutively, in any order.

Embodiment 40 Use of the expression cassette according to embodiments 1 to 29 or the vector nucleic acid according to embodiment 30 or 31 or the host cell according to embodiments 32 to 35 for the production of a polypeptide of interest.

Embodiment 41 Use of the expression cassette according to embodiments 1 to 29 or the vector nucleic acid according to embodiment 30 or 31 for improving production of a polypeptide of interest by a host cell, including introducing the expression cassette or vector nucleic acid into a host cell capable of producing the polypeptide of interest.

Embodiment 42 The use according to embodiment 41, wherein the host cell comprises a nucleic acid encoding the polypeptide of interest.

Embodiment 43 The use according to embodiment 41 or 42, wherein the expression cassette or the vector nucleic acid does not comprise a coding sequence for the polypeptide of interest.

Embodiment 44 The use according to embodiments 41 to 43, wherein improving production of a polypeptide of interest includes increasing the yield and/or increasing the purity of the polypeptide of interest.

### FIGURES

FIG. 1 shows the vector map of the original pCMV vector for expression of a polypeptide of interest comprising a heavy chain and a light chain. The vector comprises a folate receptor selectable marker gene (FAR), a DHFR selectable marker gene, and an ampicilin resistance gene.

FIG. 2 shows the vector map of the pCMV vector with included miRNA scaffold encoding the miRNA-1 into the RK intron placed in the 5′UTR of the CMV-driven transcripts of the heavy chain of POI1 (pCMV01). The intronic miRNA is highlighted.

FIG. 3 shows the vector map of the pCMV vector with included miRNA scaffold encoding the miRNA-1 into a synthetic intron placed in the 3′UTR of the SV40-driven transcripts of the FAR selectable marker gene (pCMV02). The intronic miRNA is highlighted.

FIG. 4 shows a putative mode of action and general assembly of the intronic pri-miRNA with encoded artificial miRNA targeting the receptor of POI1 placed into the RK intron. The miRNA scaffold includes two restriction sites (RE1 and RE2) for exchange of miRNA sequences. The miRNA loop structure as well as regions in the miRNA scaffold are crucial for efficient processing of miRNA molecules. The intronic region ensures the production of two functional RNA molecules: i) the intact mRNA molecule enabling the translation of the POI1 nucleotide sequence into a polypeptide sequence and ii) the intact miRNA molecule, which contains complementary regions to the endogenous mRNA of the POI1 receptor and therefore inhibiting POI1 receptor expression.

FIG. 5 shows RT-qPCR results of stable CHO pools expressing POI1 and intronic miRNAs encoding different guide strand sequences targeting the mRNA of the POI1 receptor. In both loci (RK intron and synthetic intron), control pools (no miRNA and miRNA-scrambled) showed similar expression profiles of POI1 receptor mRNA levels. Pools expressing POI1 receptor-targeting miRNA sequences placed into the pri-miRNA scaffold of the RK intron show highly efficient knockdown of POI1 receptor mRNA levels. All miRNAs reduce the expression below 25%, while miRNA-1 is most efficient with only 3.9% remaining POI1 receptor mRNA levels. In contrast, when placed into a synthetic intron in the 3′UTR of FAR, the POI1 receptor-targeting miRNA molecules were less efficient or even lost its capability to knockdown POI1 receptor mRNA levels. Also, miRNA-1 still showed strongest reduction in POI1 receptor mRNA levels to 13.9%. Shown are the mean+/−SD of three stable transfected pools per condition. One of the control pools (no miRNA) was used for normalization (set to 1).

FIG. 6 shows cell viabilities (A), viable cell densities (B) and POI1 titers (C) of pools run in fed-batch mode. The CHO pools expressing miRNA-1 in the RK intron showed highest viable cell densities as well as highest POI1 titers. Illustrated are the means+/−SD of three stable transfected pools per condition.

FIG. 7 shows cell line engineering and development strategy for knockdown approach of interfering gene product (IGP).

FIG. 8 shows cell viabilities (dotted lines) and viable cell densities of the different host cells during an optimized fed-batch. IGP knockdown pools (IGP_A and IGP_B) show similar viable cell densities as compared to stable transfected control cell lines (POI2 cell line and control gene KD). Illustrated are the means+/−SD of three stable transfected pools per condition. No replicates are shown for the POI2 and the host cell lines.

FIG. 9 shows POI2 titers in the different host cells. Titers of IGP KD pools are comparable to controls in optimized fed-batch conditions. Illustrated are the means+/−SD of three stable transfected pools per condition. No replicates are shown for the POI2 cell line.

FIG. 10 shows relative mRNA expression of POI2, IGP and a control gene. At day 10 of the optimized fed-batch, samples were taken and RNA was purified. POI2, IGP, control gene and GAPDH (housekeeping gene) transcripts were quantified using qPCR. Both IGP KD pools show significantly lower IGP transcript levels as compared to controls. Illustrated are the means+/−SD of three stable transfected pools per condition. No replicates are shown for the POI2 and the host cell lines. The POI2 and host cell lines were used for normalization (set to 1) of POI2, IGP and control genes, respectively.

FIG. 11 shows IGP protein levels quantified using an IGP ELISA. IGP KD pools reveal lower IGP protein levels in non-purified harvest at day 10 of an optimized fed-batch run. One selected pool of each condition and the POI2 cell line are illustrated. The POI2 cell line was used for normalization (set to 1).

FIG. 12 shows POI2 titers of IGP KD pools and single cell clones. IGP_A-encoding pools were used for single cell cloning. 96 clones were inoculated into a 24dwp standard fed-batch and POI2 titers were assessed. As expected, some clones show higher and others lower titers as compared to originating pools and the POI2 cell line controls.

FIG. 13 shows IGP mRNA levels of IGP KD single cell clones. IGP_A-encoding pools were used for single cell cloning. 96 clones were inoculated into a 24dwp standard fed-batch and at day 10 of the fed-batch IGP mRNA levels were quantified using qPCR. The majority of clones show significantly reduced levels of IGP transcripts as compared to the POI2 cell line control (normalized to 1).

FIG. 14 shows the characterization of the top 3 IGP KD clones as compared to POI2 cell line in a 7 L bioreactor run. IGP_A clone-A and clone-B were inoculated in two separate bioreactors, while IGP_A clone-C and the POI2 cell line were inoculated in a single bioreactor. A: POI2 titers at day 14 of the fed-batch process derived from 7 L bioreactors are significantly higher in the IPG KD clones. B: IGP mRNA levels at day 10 of standard fed-batch performed in 7 L bioreactors are greatly reduced compared to POI2 cell line (normalized to 1). IGP_A clone-B shows more than 100fold reduction of IGP transcripts. C: IGP protein levels in non-purified harvests derived from 7 L bioreactors runs show that the top 3 clones have IGP levels below the LOQ (2 ppm) as compared to the POI2 cell line.

FIG. 15 shows titer and RT-qPCR results of stable CHO pools expressing POI3 and an intronic miR-3G-derived miRNA scaffold encoding a guide strand sequence targeting the mRNA of the POI1 receptor. A: POI3 titers were similar of CHO pools producing POI3 and either encoding no miRNA or an intronic miRNA with a miR-3G-derived miRNA scaffold targeting the POI1 receptor. B: The CHO pools expressing the intronic miRNA scaffoled derived from miR-3G and targeting the POI1 receptor efficiently reduce the mRNA levels of POI1 receptor as compared to no miRNA control pools. Illustrated are the means+/−SD of three stable transfected pools per condition. One pool of the control (no miRNA) was used for normalization (set to 1).

FIG. 16 shows the triple-miRNA concept (A) and qPCR data of transfected CHO pools (B). A: The vector pCMV05 encodes an intronic miRNA cluster with three implemented artificial miRNAs each targeting a different protease. The three miRNAs are located in a single intron, separated with linkers and encoded in a single expression cassette which drives the expression of the protease-targeting miRNA cluster and the POI4 gene. B: Stable pools were generated with pCMV05-derived vectors that either encode no miRNA (POI4 pool) or triple miRNA clusters targeting protease-5, -7 and -10 (POI4-triple miR-A pool) or protease-4, -9 and -11 (POI4-triple miR-B pool). After pool generation, the expression of targeted and selected non-targeted proteases as well as of the POI4 gene were quantified using qPCR. The pools expressing triple miRNA clusters significantly reduced expression of all three targeted proteases, while non-targeted protease and POI4 gene expressions remained mainly unaffected. The targeted proteases are indicated with an arrow. Illustrated are the means+/−SD of two stable transfected pools per condition. One pool of the POI4 cell line (no miRNA) was used for normalization (set to 1).

FIG. 17 shows the 14-miR concept (A), the qPCR data of transfected CHO pools (B) and CHO clones (C). A: The vector pCMV06 encodes two intronic miRNA clusters separated in two different expression cassettes encoded in a single vector with hygromycin and puromycin selection markers. The intronic miRNA clusters encode nine and five artificial miRNAs, respectively, targeting different proteases. The nine and five miRNAs are each located in a single intron, separated with linkers and each cluster is encoded in a single expression cassette which solely drives the expression of the protease-targeting intronic miRNA cluster. B: Stable pCMV06-expressing CHO pools were generated and gene expression of targeted protease and a control gene assessed using qPCR. All 14 targeted protease genes were significantly reduced as compared to the host cell line, while the control gene expression was not impacted. Illustrated are the means+/−SD of three stable transfected pools per condition. One replicate of the duplicate host cell line was used for normalization (set to 1). C: Monoclonal cell lines derived from the pCMV06-expressing CHO pools were generated and qPCR data of three clones are shown. 14miR clone-A shows a significant knockdown of the proteases targeted by the 5miR cluster, however, exhibit no knockdown of the proteases targeted by the 9miR cluster. In contrast, 14miR clones-B and -C demonstrate a significant downregulation of all of the 14 targeted proteases. The host cell line was used for normalization (set to 1).

FIG. 18 The 14miR clones were stably transfected with a POI5-expressing vector and generated 14miR clone POI5 pools were inoculated into a standard fedbatch to assess production titers (A) and proteolytic degradation of purified and low pH- and time-stressed POI5 (B). A: The 14miR clone POI5 pools produce similar POI5 titers as compared to the host cell line POI5 pool control. B: POI5 was purified and analyzed via MS at day 0. After incubation at pH 5 for 7 days, the purified POI5 was re-analyzed using MS. POI5 material derived from 14miR clone-A POI5 pool exhibit higher proteolytic degradation as compared to the host cell line, however, both 14miR clones-B and -C show lower or no proteolytic degradation of POI5.

FIG. 19 The 14miR clones were stably transfected with a POI6-expressing vector and generated 14miR clone POI6 pools were inoculated into a standard fed batch to assess production titers (A) and proteolytic degradation of purified and low pH- and room temperature-stressed POI5 (B). A: The 14miR clone POI6 pools produce similar POI6 titers as compared to the host cell line POI6 pool control. B: POI6 was purified and analyzed via MS at day 0. After incubation at pH 5 for 7 days at RT, the purified POI6 was re-analyzed using MS. POI6 material derived from 14miR clone-A POI6 pool exhibit higher proteolytic degradation as compared to the host cell line, however, both 14miR clones-B and -C show significantly lower proteolytic degradation of POI6.

### EXAMPLES

In the following examples, different expression cassettes comprising a template sequence for a pri-miRNA were used to knock down target genes in the host cells which interfere with the production of the polypeptide of interest. In examples 1 to 4, the pri-miRNA template is present in an intronic sequence within the 5′UTR of the polypeptide of interest or within the 3′UTR of the selectable marker gene, and it targets a receptor protein of the host cell to which the polypeptide of interest binds and negatively affects the host cell's growth and survival. In example 5, a host cell already engineered to produce a polypeptide of interest is further transfected with a vector containing an expression cassette which only comprises the pri-miRNA template within an intronic sequence, but no coding sequence for a polypeptide. Here, the miRNA targets a host cell protein which is difficult to separate from the polypeptide of interest during the purification process. In example 6, an alternative miRNA scaffold is used to target a receptor protein. In example 7, a host cell is engineered with an expression cassette encoding a polypeptide of interest and containing a pri-miRNA cluster in an intronic sequence within the 5′UTR. Here, the pri-miRNA cluster contains three pri-miRNAs each targeting host cell proteins that have proteolytic functions. In example 8, a host cell is engineered with two separate expression cassettes each containing a pri-miRNA cluster, but no coding sequence for a polypeptide. The engineered cell was further transfected with a vector encoding a polypeptide of interest. Here, the miRNA targets host cell proteins that proteolytically degrade the polypeptide of interest.

### Example 1: Vector Design

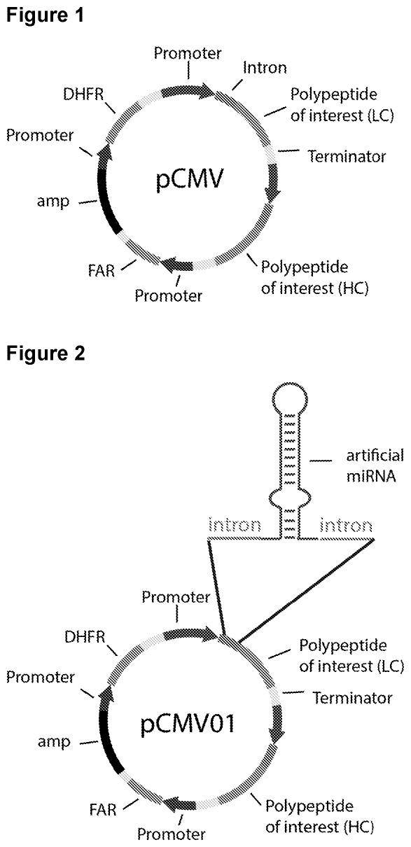

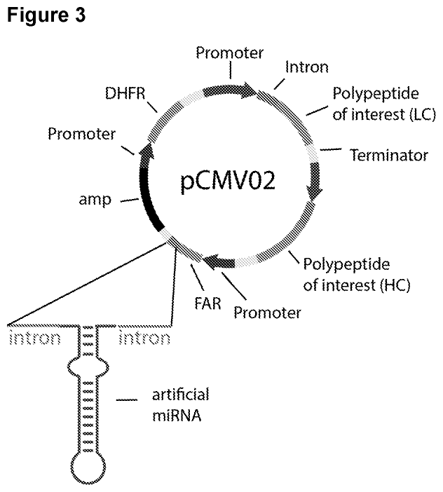

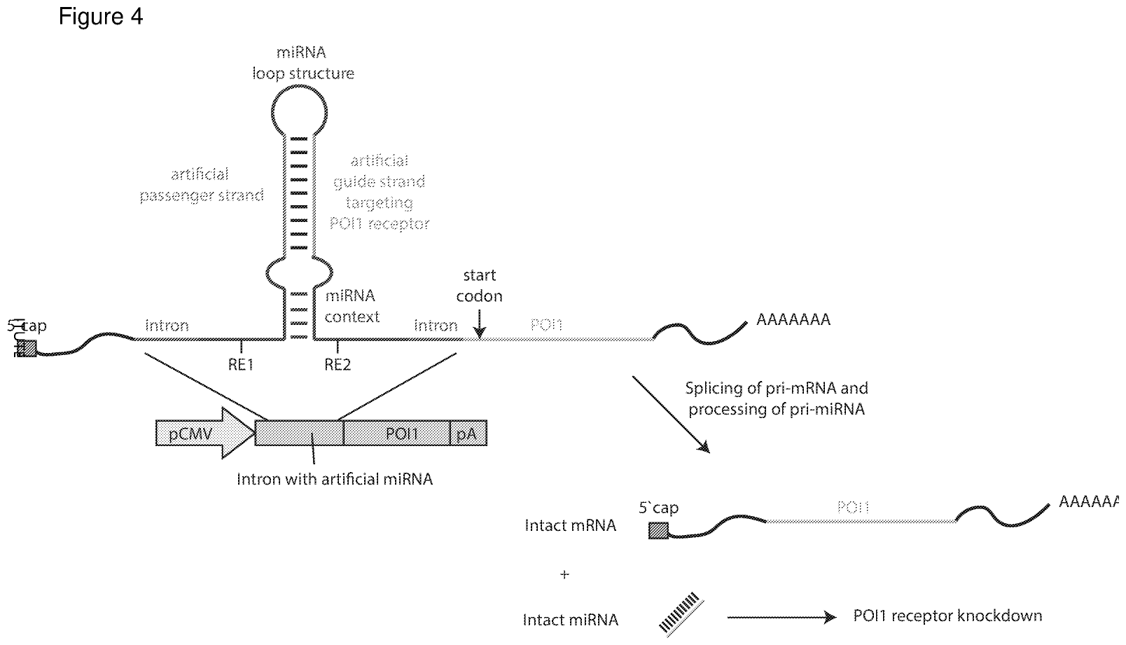

The intronic-miRNA encoding vectors (pCMV01-pCMV12) were based on a standard vector (pCMV), which encodes for CMV-driven expression of the polypeptide of interest POI1 (FIG. 1). The vector was modified by insertion of the intronic miRNA sequences into two different loci: the miRNA scaffold is either placed into an RK intron upstream of the POI1 gene (FIG. 2) or into a synthetic intron, which is implemented into the 3′ UTR of the selectable marker gene (folate receptor, FAR, FIG. 3). The sequence environment selected for the miRNA scaffold for proof of concept is similar to the human miR-30A and the miR-E molecules (e.g. Fellmann et al., 2011, Molecular Cell 41, 733-746) and is expected to lead to optimal processing of resulting miRNA sequences. However, the sequence was further modified: i) the EcoRI restriction site was replaced with BgIII restriction site, ii) the sequence downstream of the XhoI restriction site was replaced with a CHO-derived sequence and iii) additional miR-30A scaffold was added up- and downstream of the published sequence. Different miRNA sequences targeting a receptor of POI1 were tested: miRNA-1, miRNA-2, miRNA-3, miRNA-4, miRNA-5 and miRNA-sc (scrambled guide strand sequence as control). The design of the pri-miRNA is shown in FIG. 4.

### Example 2: RT-qPCR Analysis of Stable CHO Pools

To test whether POI1 receptor-targeting miRNA sequences placed into intronic pri-miRNA lead to a knockdown of POI1 receptor mRNA levels as well as to improved growth rates and higher productivity of POI1-producing CHO cells, cells were transfected with the vectors pCMV and pCMV01-12. Stable pools were selected using different selection markers and used for RT-qPCR analysis.

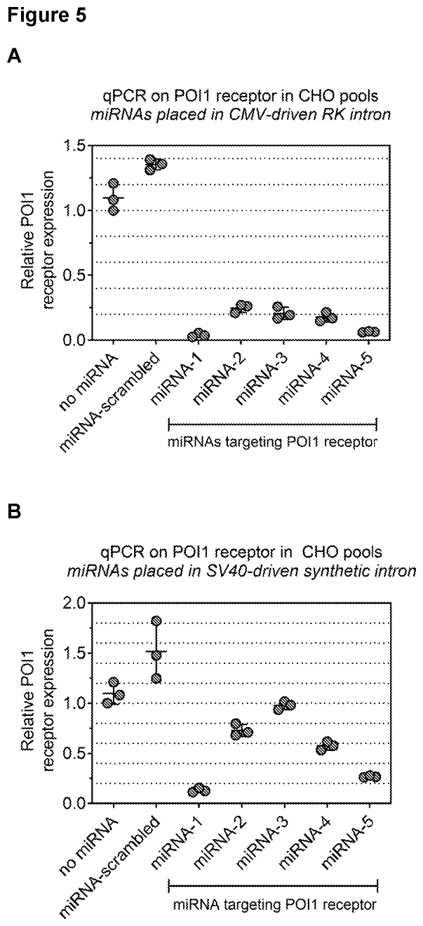

The quantification of mRNA levels of POI1 receptor using RT-qPCR revealed that all miRNA sequences targeting POI1 receptor downregulate the POI1 receptor mRNA levels when placed into the RK intron. Pools transfected with control vectors (no miRNA; miRNA-scrambled) exhibited comparable levels of POI1 receptor mRNA. The best-performing miRNA-1 reduced the POI1 receptor mRNA levels to 3.9% as compared to 109% (no miRNA; see FIG. 5A). In contrast, when miRNAs were placed into a synthetic intron in the 3′UTR of FAR, the knockdown efficiencies were less pronounced, however, miRNA-1 showed a reduction of POI1 receptor mRNA levels to 13.9% (see FIG. 5B). The differences can be explained by the locus of the intronic miRNAs: when placed into the RK intron, the transcripts are driven by the strong CMV promoter, which should simultaneously lead to higher amounts of processed miRNA molecules. In contrast, intronic miRNA expression in the 3′UTR of the FAR are driven by the weaker SV40 promoter leading to lower amounts of processed miRNA molecules as compared to the CMV promoter. These data also confirm the high knockdown efficiency of miRNA-1 when compared to other miRNA sequences, which require a stronger expression for efficient knockdown of POI1 receptor mRNA levels.

### Example 3: Cell Growth and Productivity of Stable CHO Pools

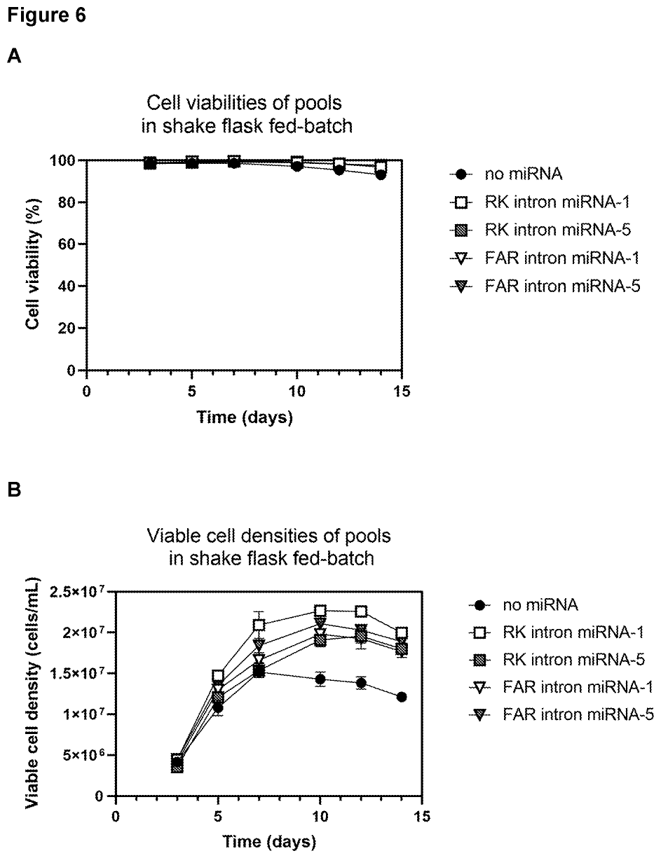

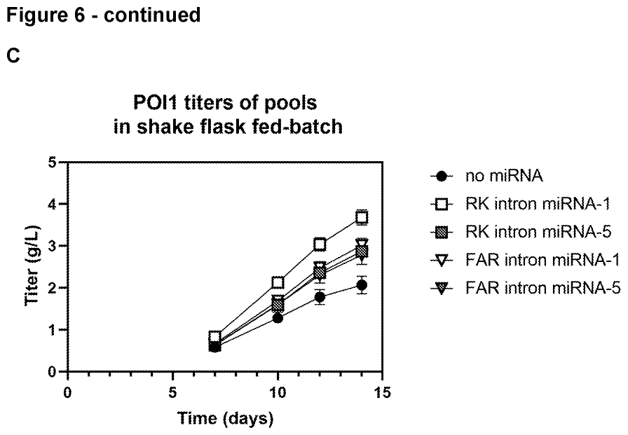

Best-performing pools (miRNA-1 and miRNA-5 in both loci, RK intron and FAR intron) as well as “no miRNA”-pools were run in fed-batch mode. Therefore, standard 14-day 100 mL shake flask fed-batch cultures were inoculated with POI1-producing pools and cell growth, cell viability as well as product titers were assessed at different time points. Pools with normal POI1 receptor mRNA levels (no miRNA) showed slower cell growth and lower viable cell densities as compared to POI1 receptor knockdown pools (FIG. 6). Stable CHO pools, which showed strongest POI1 receptor knockdown efficiency (miRNA-1 in RK intron) also revealed fastest cell growth and highest viable cell densities (FIG. 6). All POI1 receptor knockdown pools grew to higher viable cell densities as compared to the control pools. The CHO pools without miRNA revealed a titer up to 2 g/L. All POI1 receptor knockdown pools showed higher titers as compared to the control. Use of the miRNA-1 in the RK intron led to the highest titer (3.7 g/L), which is an 85% titer increase as compared to the CHO control pools (no miRNA).

### Example 4: Stability of POI1 Receptor Knockdown and POI1 Productivity in CHO Pools

We tested the stability of the POI1 receptor knockdown by the quantification of the POI1 receptor transcript levels using qPCR every four weeks. Also, we checked the POI1 titer levels in batch mode every two weeks. Interestingly, after 8 weeks, pools without POI1 receptor knockdown produce lower amounts of POI1 as compared to POI1 receptor knockdown pools. This observation might confirm the hypothesis that the knockdown of POI1 receptor gives the cell pools a survival signal. Linking the miRNA expression with the POI1 expression might stabilize the genetic integration of the pCMV cassette and therefore reduces the risk to select an unstable clone. Also, the qPCR results show that the POI1 receptor knockdown remains stable throughout 8 weeks, while the other expression cassettes of the pCMV vector do not change.

### Example 5: Interfering Gene Product (IGP) Knockdown Cell Line Generation

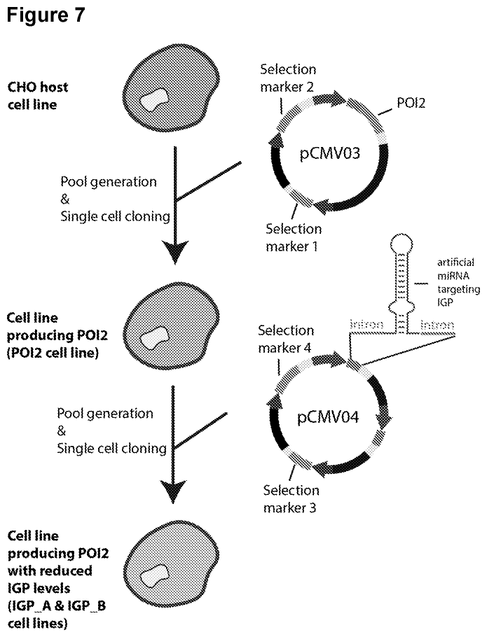

The strategy of IGP KD cell line generation is shown in FIG. 7. The CHO parental cell line was transfected with a vector encoding the polypeptide of interest POI2 and selection of pools were performed using MTX in low folate medium. The pools were going into single cell cloning and a monoclonal cell line expressing POI2 was selected, called POI2 cell line. Subsequently, the primary seed lot (PSL) of POI2 cell line was used to transfect a vector encoding the artificial intronic miRNA targeting the interfering gene product IGP and pools were generated using puromycine.

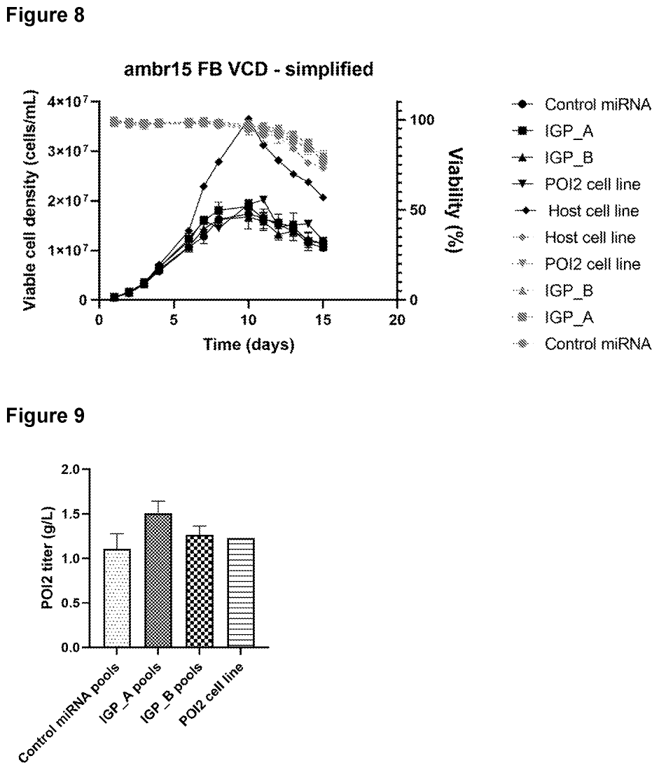

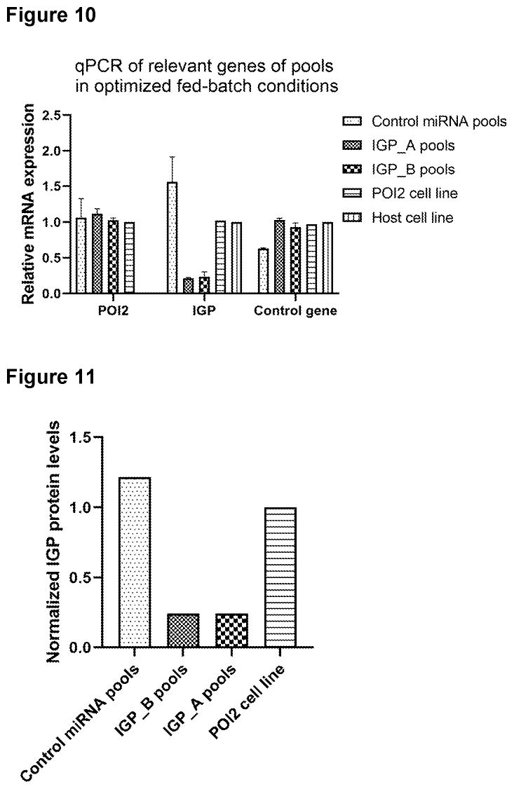

Two different miRNAs were generated targeting CHO IGP mRNA called IGP_A and IGP_B, both targeting the 3′UTR of the transcript. For pool generations, a miRNA targeting a different control gene, the parental POI2 cell line as well as the empty parental host cell line (CHO) as controls were included. All samples (triplicate pool generations for the knockdown approaches) were inoculated into an optimized fed-batch run and cell growth, gene expression and POI2 titers were assessed at different days (FIGS. 8 to 10). Also, the IGP protein levels on harvest level were assessed using an IGP ELISA (FIG. 11):

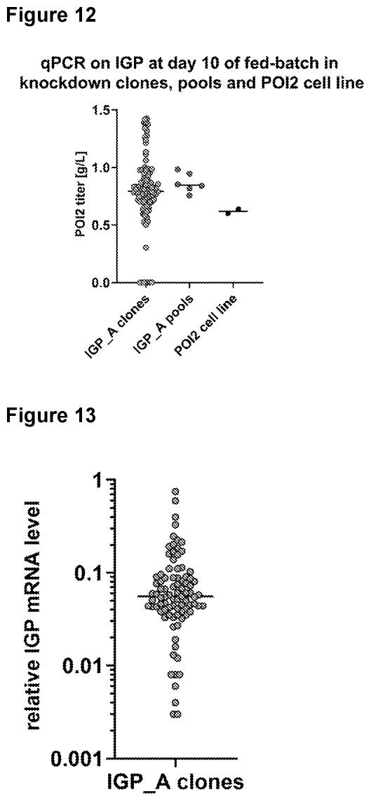

After pool generations and confirmation of an efficient IGP knockdown on mRNA level, the IGP_A pools were selected for single cell cloning and 96 expanded clones were inoculated into a 24dwp standard fed-batch to assess IGP knockdown efficiencies and POI2 productivities (FIGS. 12 and 13).

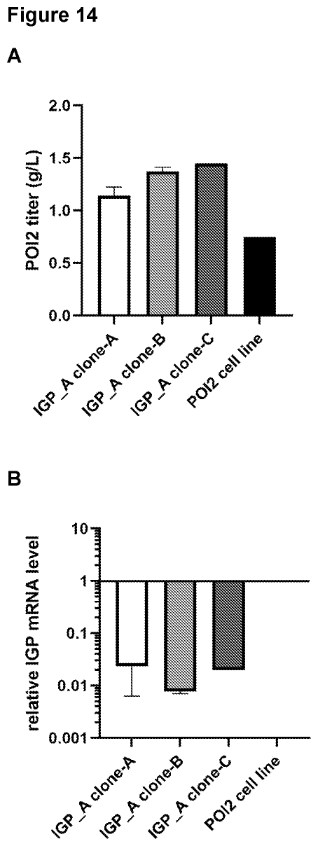

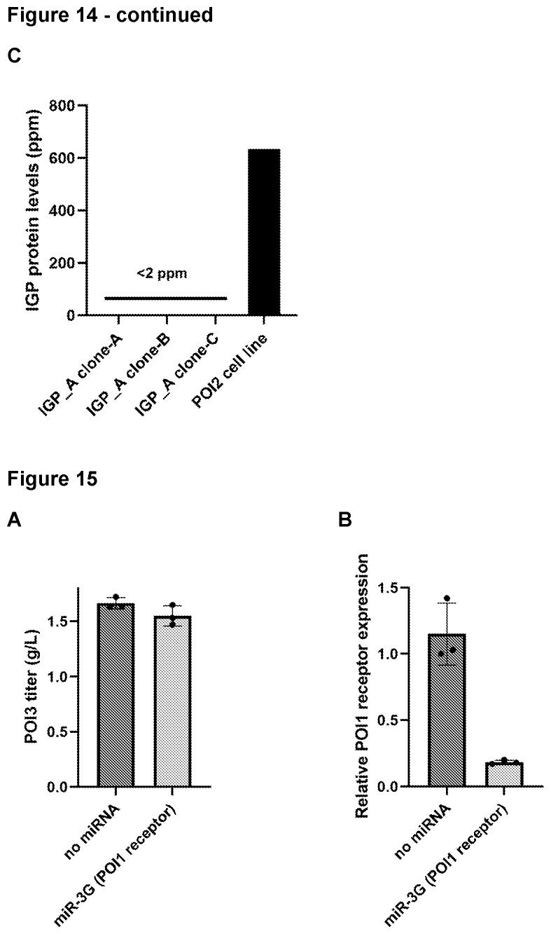

The top 30 clones were further characterized. Based on many parameters (USP, DSP, IGP data, POI2 protein characteristics) the top 3 clones were selected and inoculated into a 7 L bioreactor. IGP expression was significantly reduced in the IGP knockdown clones, resulting in an increased POI2 titer (FIG. 14).

### Example 6: Alternative miRNA Scaffold

An alternative miRNA scaffold was tested for the knockdown of POI1 receptor based on the miR-16-2 and the miR-3G sequence (e.g. Watanabe et al., 2016, RNA Biology 13 (1), 25-33). For this, the miRNA-1 was implemented into an adapted miR-3G scaffold. The artificial miRNA is encoded in a POI3-encoding expression cassette driven by a CMV promoter. Stable CHO pools were generated and POI3 titers and POI1 receptor mRNA expression were quantified. The control pool (no miRNA) produced similar titers of POI3 as compared to pools expressing miRNA-1 encoded in the adapted miR-3G scaffold (FIG. 15A). Also, the POI1 receptor was efficiently downregulated in pools expressing the miRNA-1 encoded in the adapted miR-3G scaffold (FIG. 15B).

### Example 7: Multiplexed Knockdown Strategies Using Artificial miRNA Clusters

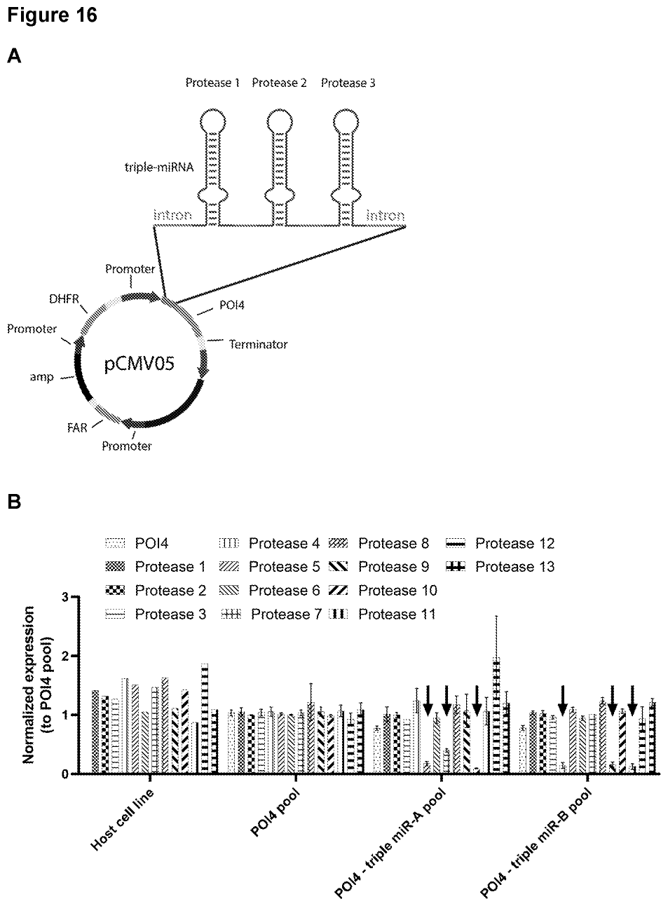

Encoding multiple miRNAs subsequently in a single intron enables simultaneous knockdown of multiple target genes. The approach was tested for a triple knockdown of three different proteases. Three miRNAs, each targeting a different protease, are separated by specific spacer sequences to ensure proper RNA folding and efficient miRNA processing. The triple miRNA cluster is implemented in an intron of a CMV-driven expression cassette driving the expression of POI4 (FIG. 16A). Two different triple miRNA clusters were designed for the knockdown of three different proteases: triple miR-A targets proteases 5, 7 and 10, while triple miR-B targets proteases 4, 9 and 11.

Stable CHO pools were generated using vectors producing POI4 and encoding either no miRNA, triple miR-A or triple miR-B. The mRNA expression of POI4, targeted and non-targeted proteases were quantified using RT-PCR and normalized to pools producing POI4 only (no miRNA) (FIG. 16B). Stable CHO pools encoding triple miR-A and triple miR-B efficiently perform knockdowns of specific target proteases, while expression of unrelated, non-targeted proteases remain similar to control pools.

### Example 8: 14miR Protease Knockdown Cell Line Generation

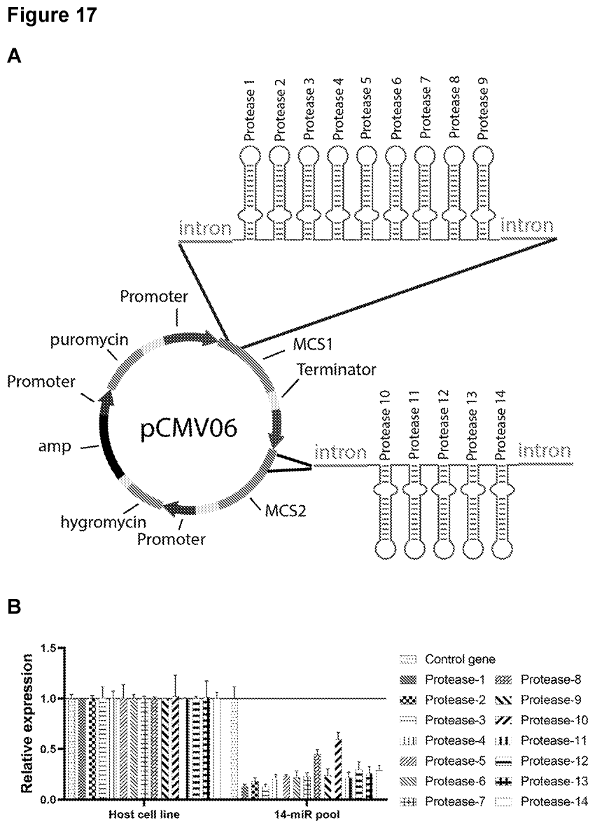

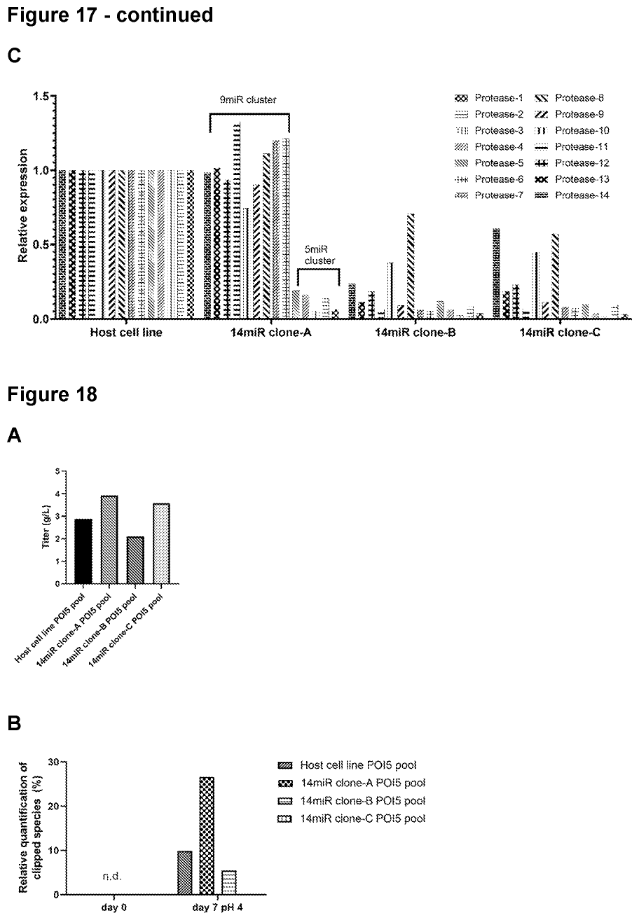

The 14miR-encoding vector pCMV06 comprises of two expression cassettes driving the expression of an intronic miRNA cluster, either targeting nine or five endogenous proteases, but no coding sequence for a polypeptide of interest (FIG. 17A). The selection markers puromycin and hygromycin are encoded on separate expression cassettes allowing selection of stable CHO pools using either of the selection markers. Stable CHO pools expressing pCMV06, called 14-miR pool, were generated using puromycin as selection marker. The expression of the targeted endogenous proteases was significantly reduced in 14-miR pools compared to the host cell line as measured using qPCR (FIG. 17B). However, the expression of a non-targeted, endogenous control gene remained unaffected. Monoclonal 14-miR clones were generated using single cell cloning and three clones, called 14miR clone-A, -B and -C were further characterized using qPCR (FIG. 17C). 14miR clone-A downregulate the proteases targeted by the 5miR cluster, but not the proteases targeted by the 9miR cluster assuming the loss of the 9miR expression cassette. In contrast, 14miR clones-B and -C significantly reduce the expression of all 14 targeted endogenous proteases. Most of the proteases were strongly downregulated by the miRNA clusters, while two to three miRNAs exhibit a modest knockdown.

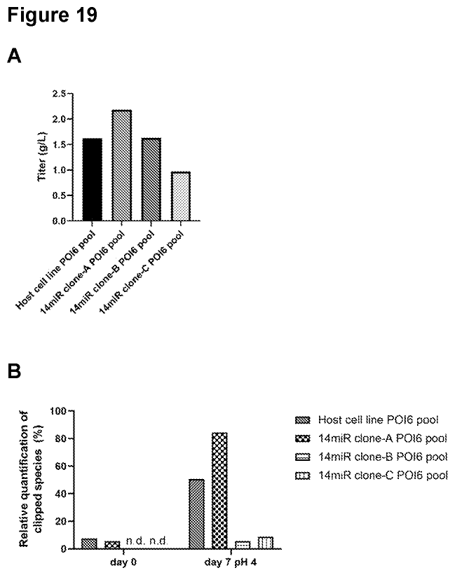

The 14miR clones were stably transfected with a vector encoding for POI5 or POI6. The host cell line was transfected as a control. Stable pools were generated using MTX in low folate medium. The generated 14miR clone POI5 or POI6 pools as well as the host cell line POI5 or POI6 pools were inoculated into a standard fed-batch and production titers were assessed at day 14. The 14miR clone POI5 and POI6 pools showed similar titers of POI5 or POI6 as compared to the host cell line pools (FIGS. 18A and 19A). A clone-to-clone variation regarding the productivity of the polypeptides of interest can be observed. Also, production harvest was used to purify POI5 or POI6 and purified material was analyzed using mass spectrometry at day 0. The purified material was exposed to low pH for 7 days at room temperature and re-analyzed using mass spectrometry (FIGS. 18B and 19B). The proteolytic degradation after the 7 days treatment of POI5 or POI6 was higher in the 14miR clone-A pools as compared to the host cell line pools. In contrast, the 14miR clone-B and -C exhibit significantly lower proteolytic degradation as compared to the host cell line pools.

### Example 9: Materials and Methods

### 1. Expression Vector Construction

The vectors used in the examples consist of following elements: hCMV promoter/enhancer driving expression of the individual genes needed for assembly of the POI constructs, polyadenylation signal (polyA), folic acid receptor, DHFR, puromycin and hygromycin resistance genes as selection markers, E.Coli origin (ColE ori) of replication and the beta-lactamase gene for ampicillin (amp) resistance to enable amplification in bacteria. Different plasmid setups were evaluated and more details are provided within the figures.

### 2. Cell Lines, Cultivation, Transfection and Selection

CHO cell lines were cultivated in 24-deep well plates or shake flasks in a non-humidified shaker cabinet at 300 rpm (24dwp) or 150 rpm (shake flasks), 10% CO₂ at 36.5° C. in suspension in proprietary, chemically defined culture media. Cell viabilities and growth rates were monitored by means of an automated system (ViCell, Beckman Coulter) or using an analytical flow cytometry (CytoFlex, Beckman Coulter). Cells were passaged 2-3 times per week into fresh medium and were maintained in logarithmic growth phase.

Linearized expression vectors were transfected by electroporation (Amaxa Nucleofection system, Lonza, Germany). The transfection reaction was performed in chemically defined cultivation medium, according to the manufactures instructions. The parental CHO cells used for transfection were in exponential growth phase with cell viabilities higher than 95%. Transfections were performed with 5×10⁶ cells per transfection. Immediately, after transfection cells were transferred into shake flasks, containing chemically defined cultivation medium. Cell pools were incubated for 48 hours at 36.5° C. and 10% CO₂ before starting the selection process.

A selection procedure was carried out using the selection markers encoded by the individual expression vectors, as described above. The proteins FoIR and DHFR are participating in the same molecular pathway; the FoIR is transporting folic acid as well as the folate analogue MTX into the cell, the DHFR is converting it into vital precursors for purine and methionine synthesis. Combining them as selective principle, a particular strong selective regime can be taken to enrich for recombinant cells expressing both recombinant protein. Puromycin selection is driven by its inhibition of protein synthesis and vectors encoding the puromycin resistance marker gene enable cells to survive in presence of puromycin.

48 h after transfection and growth under low folate conditions, additional selective pressure was applied by adding 10 nM MTX to the chemically defined cultivation medium. Alternatively, puromycin was used as selection agent. 48h after transfection 0.003 mg/mL puromycin was added to the chemically defined cultivation medium. After pool recovery cells were frozen in culture medium, supplemented with 7.5% DMSO and cell pellets prepared.

### 3. Gene Expression Analysis by Quantitative Real-Time PCR

RNA extraction was performed using the Qiagen RNeasy Mini Kit according to the manufactures instructions. For real-time qPCR, cDNA was synthesized from 200 ng/μl diluted RNA using the High Capacity RNA-to-cDNA Master Mix (Applied Biosystems) and 10× diluted cDNAs were analyzed in triplicates using the QuantiFast SYBR Green PCR Kit (Qiagen) or TaqMan Primer/Probe system and TaqMan Mastermix (Applied Biosystems). As endogenous control for normalization GAPDH was amplified. Amplification and analysis was performed using the ABI PRISM® 7900HT Sequence Detection System. For calculation of relative quantities (RQ) of gene expression for sample comparison the comparative 2^{-ΔΔCt} method was used and the data normalized.

### 4. Upstream Processing

Subsequent to selection, material was produced either in shake flask fed batch, 24-deep well plate cultures or ambr15 bioreactors. Fed batch cultures were inoculated with a cell seeding density of 4E5 vc/ml (addition of proprietary feed solutions starting on day 3 and cultivation temperature shift to 33° C. on day 5). During the cultivation in-process controls were performed to monitor the concentration of the POI constructs. Cell culture samples for RNA isolation were taken at day 10 of the process. The individual culture was cultivated over a period of 14 days. At the end of the cultivation process cells were separated from the culture supernatant by centrifugation followed by sterile filtration before further downstream processing.

### 5. Protein Quantification Using ELISA

The amount of Chinese hamster (CHO) IGP was determined using a sandwich ELISA. Samples were added to microtiter plates coated with anti-IGP antibody (capture antibody). Bound IGP is then quantified by incubation with biotinylated anti-IGP antibody (detection antibody), followed by streptavidin-peroxidase and tetramethylbenzidine (TMB) as substrate and measuring absorbance at 450 nm. The IGP levels in samples were calculated based on the CHO IGP standard.

### 6. Purification Method

Recombinant proteins were purified by chromatographic methods on an Akta avant 25 system (Cytiva). Proteins were captured by affinity chromatography at neutral pH conditions and eluted at acidic conditions with 50 mM acetic acid at pH 3.0. All eluates were up-titrated to pH 5.0 with 1 M Tris base right after elution.

### 7. Sample Treatment and Analytical Analysis

Sample buffer was exchanged using Amicon Ultra-4 Centrifugal Filter Devices. The pH of the buffer was set to pH 4 with 50 mM acetic acid. The samples were transferred into 1.5 mL Eppendorf tubes and incubated for 7 days at room temperature.

Deglycosylation of samples were performed using 1 mg of purified recombinant proteins in Tris-HCl buffer at pH7.5. PNGase F was added and incubated at 37° C. overnight.

All candidates were analyzed on a LC/MS system (WATERS, Xevo XS). The mobile phases were: (A) 0.1% Trifluoroacetic acid (TFA) in miliQ water and (B) 0.09% TFA in acetonitrile. The gradient of mobile phase B was from 5% to 50% over 7 min and total runtime was 10 min for each injection. The separation of protein degradation product was carried out (70° C.) using the Waters BioResolve RP mAb Polyphenyl Column, 450 Å, 2.7 μm, 2.1 mm×150 mm. The loading on the column for each injection was 1.0 μg for the intact analysis and 0.44 ug for the reduced analysis. The capillary voltage was set at 1.8 kV, sampling cone at 190 V and the source office offset at 30 V for all analyses. The source temperature and desolvation temperature were maintained at 125° C. and 400° C. respectively. The desolvation gas flow was at 800 L/h, cone gas at 50 L/h, and nebulizer gas at 6.5 Bar. The system was controlled by MassLynx. All data were imported to and then processed within Genedata MS Refiner workflow.

SEQUENCE LISTING

| SEQ ID | | |
|---|---|---|
| NO | Description | Sequence |
| 1 | 5′ miRNA scaffold stem | GUGCUCGACUAGGGAUAACAGGGUAAUUGUUUGAAUGAGGCUUCA |
| | | GUACUUUACAGAAUCGUUGCCUGCACAUCUUGGAAACACUUGCUG |
| | | GGAUUACUUCAGCUCUUUAACCCAACAGAAGGCUCGAGAGAAAGC |
| | | AUAUCUGUUGACAGUGAGCG |
| 2 | 5′ miRNA scaffold stem | GUGCUCGACUAGGGAUAACAGGGUAAUUGUUUGAAUGAGGCUUCA |
| | | GUACUUUACAGAAUCGUUGCCUGCACAUCUUGGAAACACUUGCUG |
| | | GGAUUACUUCAGCUCUUUAACCCAACAGAAGGCUCGAGGCUAGCG |
| | | CCGAUAUAACGGCGGAGAAAGCAUAUCUGUUGACAGUGAGCG |
| 3 | 5′ miRNA scaffold stem | GUGCUCGACUAGGGAUAACAGGGUAAUUGUUUGAAUGAGGCUUCA |
| | | GUACUUUACAGAAUCGUUGCCUGCACAUCUUGGAAACACUUGCUG |
| | | GGAUUACUUCACCUGUUUAACCCAACAGAAGGCUUAAGAGAAAGC |
| | | AUAUCUGUUGACAGUGAGCG |
| 4 | 5′ miRNA scaffold stem | GUGCUCGACUAGGGAUAACAGGGUAAUUGUUUGAAUGAGGCUUCA |
| | | GUACUUUACAGAAUCGUUGCCUGCACAUCUUGGAAACACUUGCUG |
| | | GGAUUACUUCGACUUUUCUAACCAACAGAAGGCGUACGAGAAAGC |
| | | AUAUCUGUUGACAGUGAGCG |
| 5 | 5′ miRNA scaffold stem | GUGCUCGACUAGGGAUAACAGGGUAAUUGUUUGAAUGAGGCUUCA |
| | | GUACUUUACAGAAUCGUUGCCUGCACAUCUUGGAAACACUUGCUG |
| | | GGAUUACUUCGACUUCUUAACCCAACAGAAGGCUCGAGAAGGUAU |
| | | AUUGCUGUUGACAGUGAGCG |
| 6 | 5′ miRNA scaffold stem | CUCGAGCCGGAUCAACGCCCUAGGUUUAUGUUUGGAUGAACUGAC |
| | | AUCCGCGUAUCCGUC |
| 7 | 5′ miRNA scaffold stem | CUCGAGCAGCCAGCUUUUUGCGAAUCUCGACA |
| 8 | miRNA scaffold loop | UAGUGAAGCCACAGAUGUA |
| 9 | miRNA scaffold loop | GUAGUGAAAUAUAUAUUAAAC |
| 10 | miRNA scaffold loop | UGUGUUUUUUUUGAA |
| 11 | 3′ miRNA scaffold stem | UGCCUACUGCCUCGGACUUCAAGGGGCUAAGAUCUGGCAAUUAUC |
| | | UUGUUUACUAAAACUGAAUACCUUGCUAUCUCUUUGAUACAUUUU |
| | | UACAAAGCUGAAUUAAAAUGGUAUAAAUUAAAUCACUUUUUU |
| 12 | 3′ miRNA scaffold stem | UGCCUACUGCCUCGGACUUCAAGGGACUAGUAGAUCUCAAUUAUC |
| | | UUGUUUACUAAAACUGAAUACCUUGCUAUCUCUUUGAUACAUUUU |
| | | UACAAAGCUGAAUUAAAAUGGUAUAAAUUAAAUCACUUUUUU |
| 13 | 3′ miRNA scaffold stem | UGCCUACUGCCUCGGACUUCAAGGGGCUAACCGGUGGCAAUUAUC |
| | | UUGUUUACUAAAACUGAAUACCUUGCUAUCUCUUUGAUACAUUUU |
| | | UACAAAGCUGAAUUAAAAUGGUAUAAAUUAAAUCACUUUUUU |
| 14 | 3′ miRNA scaffold stem | UGCCUACUGCCUCGGACUUCAAGGGGCUAUUCGAAGCAAUUAUCU |
| | | UGUUUACUAAAACUGAAUACCUUGCUAUCUCUUUGAUACAUUUUU |
| | | ACAAAGCUGAAUUAAAAUGGUAUAAAUUAAAUCACUUUUUU |
| 15 | 3′ miRNA scaffold stem | UGCCUACUGCCUCGGACUUCAAGGGGCUAGAAUUCGGCAAUUAUC |
| | | UUGUUUACUAAAACUGAAUACCUUGCUAUCUCUUUGAUACAUUUU |
| | | UACAAAGCUGAAUUAAAAUGGUAUAAAUUAAAUCACUUUUUU |
| 16 | 3′ miRNA scaffold stem | UACGGUAACGCGGAAUACGCAACUAUUUUAUCAAUUUUUUGCGUC |
| | | GACAGACUC |
| 17 | 3′ miRNA scaffold stem | UCGCGAUUCGCUUUUUCGUCUUUGAGAUCU |
| 18 | synthetic intron with | GGAACGGUGCAUUGGAACGCGGAUUCCCCGUGCCAAGAGUGACGU |
| | splice donor | AAGUACCGCCU |
| 19 | synthetic intron with | AUAGAGUCUAUAGGCCCACCCCCUUGGCUUCGUUAGAACGCGGCU |
| | splice acceptor | ACAAUUAAUACAUAACCUUAUGUAUCAUACACAUACGAUUUAGGU |
| | | GACACUAUAGAAUAACAUCCACUUUGCCUUUCUCUCCACAGGUGU |
| | | CCACUCCCAGGUCCAACUGC |
| 20 | synthetic intron with | CAGUUCGAAGAGGUAAGU |
| | splice donor | |
| 21 | synthetic intron with | UACUAACUCUUCUUUUUUUUUUUCACAGGACCAUCGAUCGAA |
| | splice acceptor | |
| 22 | spacer sequence | CCGCCGAUAUAACGGCGG |

1. An expression cassette for expression of an miRNA in a host cell, comprising an intronic sequence comprising a template sequence for a pri-miRNA, wherein the pri-miRNA is suitable to be processed in the host cell to form a miRNA targeting a gene product of the host cell which interferes with the production of and/or modulates a polypeptide of interest recombinantly expressed in the host cell; and wherein the miRNA comprises a passenger strand and a guide strand having an artificial sequence.

2. The expression cassette according to claim 1, further comprising
(i) a polymerase II promoter and a terminator functionally linked to the template sequence for a pri-miRNA, wherein the template sequence for the pri-miRNA is located between promoter and terminator of the expression cassette; and/or
(ii) a splice donor site upstream of the pri-miRNA and a corresponding splice acceptor site downstream of the pri-miRNA.

3. The expression cassette according to claim 1, comprising two or more template sequences for a pri-miRNA, each miRNA targeting the same or a different gene product, wherein the two or more template sequences for a pri-miRNA optionally are located within the same intronic sequence

4. The expression cassette according to claim 1, wherein the pri-miRNA comprises, from 5′ to 3′, a 5′ miRNA scaffold stem, a passenger strand, a miRNA scaffold loop, a guide strand, and a 3′ miRNA scaffold stem; wherein the 5′ miRNA scaffold stem comprises the nucleotide sequence of any one of SEQ ID NOs: 1-4, and/or the miRNA scaffold loop comprises the nucleotide sequence of SEQ ID NO: 8, and/or the 3′ miRNA scaffold stem comprises the nucleotide sequence of any one of SEQ ID NOs: 11-14.

5. The expression cassette according to claim 1, wherein the gene product of the host cell targeted by the miRNA
(i) is selected from the group consisting of a protease which is capable of cleaving the polypeptide of interest, a protein involved in posttranslational modification of the polypeptide of interest, a receptor or binding partner of the polypeptide of interest, a protein which is difficult to separate from the polypeptide of interest, a protein involved in folding and/or secretion of the polypeptide of interest, a protein involved in transport of components necessary for production or modification of the polypeptide of interest, a protein involved in degradation of the polypeptide of interest, a protein which shares a sequence identity of at least 70%, in particular at least 80%, with the polypeptide of interest over its entire length, and an endogenous homologue of the polypeptide of interest;
(ii) is a protease which is capable of cleaving the polypeptide of interest;
(iii) is a transferase which is capable of catalyzing post-translational modification of the polypeptide of interest, for example acetylation, acylation, sulfation, phosphorylation, alkylation, hydroxylation, amidation, carboxylation, palmitoylation, myristoylation, or isoprenylation;
(iv) is an enzyme which is capable of catalyzing the removal of a post-translational modification or of a chemical group of the polypeptide of interest, for example a hydrolase such as a lipase, a phosphatase, or a glycosydase; or
(v) is a protein involved in glycosylation of the polypeptide of interest, in particular a glycosyltransferase, a glycosidase, or a nucleotide sugar transporter, for example a fucosyltransferase, or a sialyltransferase; and/or
(vi) is an endogenous gene product of the host cell.

6. The expression cassette according to claim 1, wherein the artificial sequence of the passenger strand and/or of the guide strand is not found in naturally occurring miRNAs.

7. The expression cassette according to claim 1, wherein the promoter is selected from the group consisting of cytomegalovirus (CMV) promoter, simian virus 40 (SV40) promoter, ubiquitin C (UBC) promoter, elongation factor 1 alpha (EF1A) promoter, phosphoglycerate kinase (PGK) promoter, Rous sarcoma virus (RSV) promoter, BROAD3 promoter, murine rosa 26 promoter, pCEFL promoter, chicken β-actin promoter (CBA), β-actin promoter coupled with CMV early enhancer (CAGG), α-1-antitrypsin promoter, and inducible promoters such as tetracycline-inducible promoters (e.g. pTRE), and vanillic acid inducible promoters; preferably a CMV promoter or a SV40 promoter.

8. The expression cassette according to claim 2, further comprising a coding sequence for the polypeptide of interest or for a selectable marker, functionally linked to the polymerase II promoter and the terminator.

9. The expression cassette according to claim 1, wherein the host cell is a mammalian cell, in particular a human, primate or rodent cell, especially a human or hamster cell, preferably a CHO cell.

10. A vector nucleic acid for transfection of a host cell, comprising the expression cassette according to claim 1.

11. The vector nucleic acid according to claim 10, further comprising an additional expression cassette suitable for expressing the polypeptide of interest.

12. A host cell comprising the expression cassette according to claim 1, wherein the host cell is capable of recombinantly expressing the polypeptide of interest.

13. The host cell according to claim 11, being a mammalian cell, in particular a human, primate or rodent cell, especially a human or hamster cell, preferably a CHO cell.

14. A method for producing a polypeptide of interest in a host cell, comprising the steps of
(a) providing a host cell according to claim 12;
(b) cultivating the host cell in a cell culture under conditions which allow for the expression of said polypeptide of interest;
(c) obtaining said polypeptide of interest from the cell culture; and
(d) optionally processing the polypeptide of interest;
wherein the polypeptide of interest may optionally be encoded on the same vector nucleic acid, especially within the same expression cassette, as the pri-miRNA.

15. The method according to claim 14, wherein step (d) comprises providing a pharmaceutical formulation comprising the polypeptide of interest.

16. A method of increasing the yield and/or increasing the purity of a polypeptide of interest produced by a host cell, comprising the steps of
(a1) providing a host cell capable of producing the polypeptide of interest;
(a2) introducing a vector nucleic acid according to claim 10 into the host cell;
(b) cultivating the host cell in a cell culture under conditions which allow for the expression of said polypeptide of interest;
(c) obtaining said polypeptide of interest from the cell culture; and
(d) optionally processing the polypeptide of interest.

17. A method for producing a host cell according to claim 12, comprising the steps of
(a) introducing a vector nucleic acid according to the second aspect into a host cell, wherein the vector nucleic acid comprises a coding sequence for the polypeptide of interest, either within the expression cassette which expresses the miRNA, or within a further expression cassette; or
(b) introducing a vector nucleic acid according to the second aspect into a host cell, wherein the vector nucleic acid does not comprise a coding sequence for the polypeptide of interest, and introducing a further vector nucleic acid suitable for recombinant expression of the polypeptide of interest into the host cell, wherein the different vector nucleic acids may be introduced into the host cell simultaneously or consecutively, in any order.

18. Use of the expression cassette according to claim 1 for the production of a polypeptide of interest.

19. Use of the expression cassette according to claim 1 for improving production of a polypeptide of interest by a host cell, including introducing the expression cassette or vector nucleic acid into a host cell capable of producing the polypeptide of interest.

20. A host cell comprising the expression cassette according to the vector nucleic acid according to claim 10, wherein the host cell is capable of recombinantly expressing the polypeptide of interest.
